(19) United States
(12) Patent Application Publication
(10) Pub. No.: US 2024/0245707 A1
(43) Pub. Date: Jul. 25, 2024

(54) COMPOSITIONS FOR TREATING INSOMNIA AND USES THEREOF

(71) Applicant: Zhejiang Jiachi Development Pharmaceuticals LTD., Hangzhou (CN)

(72) Inventor: Jun YANG, Hangzhou (CN)

(21) Appl. No.: 18/563,321

(22) PCT Filed: May 23, 2022
(86) PCT No.: PCT/CN2022/094332

§ 371 Date: Nov. 21, 2023

(30) Foreign Application Priority Data
May 25, 2021 (WO) PCT/CN2021/095700

(51) Int. Cl. A61K 31/567; A61K 9/28; A61P 25/20

(52) U.S. Cl. CPC A61K 31/567; A61K 9/28; A61P 25/20

(57) ABSTRACT

It provides methods and compositions for treating insomnia comprising administering a composition comprising a compound of formula A or formula I (e.g., anordrin) or a pharmaceutically acceptable salt or ester thereof. In some embodiments, the compound is anordrin.

### CROSS-REFERENCE TO RELATED APPLICATION

This application claims priority to International application PCT/CN2021/095700, filed on May 25, 2021, the content of which is incorporated by reference in their entirety for all purposes.

### FIELD OF THE INVENTION

The present invention relates to methods and compositions of treating insomnia.

### BACKGROUND OF THE INVENTION

Sleep is essential for health and quality of life. Insomnia is a growing health problem around the world. Insomnia is a condition characterized by difficulty falling asleep (sleep onset), waking frequently during the night (fragmented sleep), waking too early (premature final awakening), and/or waking up feeling un-refreshed. In the National Sleep Foundation's (NSF) Sleep in America Poll 2005, 42% of survey respondents reported that they awoke frequently during the night, 22% of adults reported waking too early and not being able to return to sleep and 38% reported waking and feeling un-refreshed.

Estrogen is a hormone that regulates human reproductive development and metabolism. For example, women subject to oophorectomy and post-menopausal symptoms who have decreased blood estrogen levels develop autonomous organ dysfunction or atrophy symptoms. Most notably, these include hot flashes, mood disorders, insomnia and sleep-disordered breathing. Generally, post-menopausal women are less satisfied with their sleep and as many as 61% report insomnia symptoms.

Treatment with estrogen (Estrogen Replacement Therapy, ERT) or with estrogen and progesterone (Hormone Replacement Therapy, HRT) has been found to help relieve menopausal symptoms. However, recent large-scale U.S. government funded studies, the Women's Health Initiative, were stopped due to safety concerns since it was found that taking HRT may put women at risk for cardiovascular disease and dementia. It was also widely recognized that estrogen treatment can cause hormone-induced cancer such as breast cancer and endometrial cancer.

The disclosures of all publications, patents, patent applications and published patent applications referred to herein are hereby incorporated herein by reference in their entirety.

### BRIEF SUMMARY OF THE INVENTION

The present application provides methods of treating insomnia in an individual, comprising administering to the individual an effective amount of a composition comprising a compound of formula I (e.g., anordrin) as described herein, or a pharmaceutically acceptable salt or ester thereof. In some embodiments, the compound is anordrin.

In one aspect of the present application, there is provided a method of treating insomnia in an individual, comprising administering to the individual an effective amount of a composition comprising a compound of formula I:

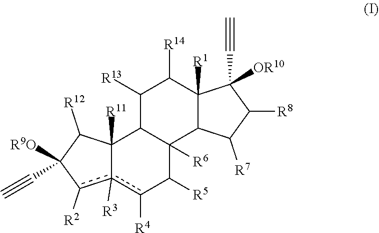

or a pharmaceutically acceptable salt or ester thereof, wherein: a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or [symbol] between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl; b) R¹ is C₁-C₃ alkyl; c) R⁵, R⁷, R⁸, R¹², R¹³ and R¹⁴ are independently selected from the group consisting of hydrogen, hydroxyl, mercaptan, keto, and C₁-C₃ alkyl; d) R⁶ is selected from the group consisting of hydrogen, hydroxyl, mercaptan, and C₁-C₃ alkyl; e) R⁹ is selected from the group consisting of hydrogen and —C(═O)R^{9a}; f) R¹⁰ is selected from the group consisting of hydrogen and —C(═O)R^{10a}; g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and h) R¹¹ is selected from the group consisting of C₁-C₃ alkyl. In some embodiments, R⁹ and R¹⁰ are independently hydrogen or —C(═O)CH₂CH₃. In some embodiments, one or both of R¹ and R¹¹ are —CH₃. In some embodiments, any one or more of R², R³, R⁴, R⁵, R⁶, R⁷, R⁸, R¹², R¹³, and R¹⁴ are hydrogen.

In some embodiments according to any one of the methods described above, the compound is anordrin.

In some embodiments according to any one of the methods described above, the individual is a female.

In some embodiments according to any one of the methods described above, the individual is a human.

In some embodiments according to any one of the methods described above, the individual is at perimenopause stage, reaches menopause, is post menopause, or has been subject to a surgical removal of ovary. In some embodiments, the individual is a postmenopausal woman.

In some embodiments according to any one of the methods described above, the individual has a low level of estrogen.

In some embodiments according to any one of the methods described above, the individual has a score of at least about 7 according to Athens Insomnia Scale.

In some embodiments according to any one of the methods described above, the individual has a score of at least about 11 according to Pittsburgh sleep quality index (PSQI).

In some embodiments according to any one of the methods described above, the insomnia is assessed with other sleep disorder questionnaire (SDQ) such as Epworth Sleeping Scale. In some embodiments, the insomnia is assessed with a sleeping monitor.

In some embodiments according to any one of the methods described above, the individual is not subject or has not been subject to a hormone therapy.

In some embodiments according to any one of the methods described above, the dose of the compound for each administration is at least about 20 μg for a human or a comparable dose for an individual that is not a human. In some embodiments, the dose of the compound for each administration is at least about 50 μg for a human or a comparable dose for an individual that is not a human. In some embodiments, the dose of the compound for each administration is at least about 100 μg for a human or a comparable dose for an individual that is not a human. In some embodiments, the dose of the compound for each administration is at least about 150 μg for a human or a comparable dose for an individual that is not a human.

In some embodiments according to any one of the methods described above, the dose of the compound for each administration is no more than 1 mg for a human or a comparable dose for an individual that is not a human.

In some embodiments according to any one of the methods described above, the dose of the compound for each administration is about 20 μg to about 2 mg for a human or a comparable dose for an individual that is not a human. In some embodiments, the dose of the compound for each administration is about 20 μg to about 200 μg for a human or a comparable dose for an individual that is not a human. In some embodiments, the dose of the compound for each administration is about 400 μg to about 2 mg for a human or a comparable dose for an individual that is not a human.

In some embodiments according to any one of the methods described above, the composition is administered at a frequency of about once every two weeks to about three times a day. In some embodiments, the composition is administered daily. In some embodiments, the composition is administered daily at a dosage of about 20 μg to about 200 μg (such as about 50 μg, about 100 μg, or about 150 μg). In some embodiments, the composition is administered weekly. In some embodiments, the composition is administered weekly at a dosage of about 400 μg to about 2 mg.

In some embodiments according to any one of the methods described above, the composition is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the composition is administered orally.

In some embodiments according to any one of the methods described above, the method further comprises administering a second agent.

In some embodiments according to any one of the methods described above, the method further comprises assessing estrogen level in the individual.

In some embodiments according to any one of the methods described above, the insomnia is chronic insomnia.

In another aspect of the present application, there is provided a unit dosage form for treating insomnia comprising a) a composition comprising a compound of formula I, wherein the amount of the compound is in the range of about 20 μg to about 2 mg, and b) a pharmaceutical acceptable carrier.

In another aspect of the present application, there is provided a use of an effective amount of a composition comprising a compound of formula I (e.g., anordrin) for treating insomnia.

In another aspect of the present application, there is provided a composition comprising a compound of formula I (e.g., anordrin) for treating insomnia.

In another aspect of the present application, there is provided a kit comprising a) a composition comprising a compound of formula I, and b) instructions for using the kit for treating insomnia.

### BRIEF DESCRIPTION OF THE DRAWINGS

FIG. 1 depicts an exemplary diagnosis procedure for chronic insomnia.

FIG. 2 depicts an exemplary table for self-assessment of insomnia using Athens Insomnia Scale (AIS).

FIG. 3 depicts change of AIS number after treatment with anordrin.

FIGS. 4A-4B depict various formulas exemplified in the present application.

FIG. 5 depicts that anordrin induces ATP surge in the culture of neuronal cells.

### DETAILED DESCRIPTION OF THE INVENTION

The present application provides methods and compositions for treating insomnia. In some embodiments, the present application provides a composition and a method of using the composition for treating insomnia in an individual, wherein the composition comprises a compound of formula A:

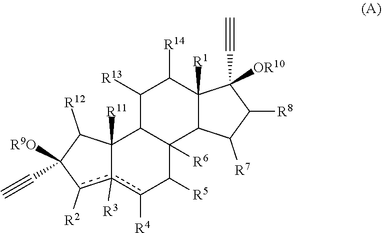

or a pharmaceutically acceptable salt or ester thereof, wherein: a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or [symbol] between the carbon bearing R² and the carbon bearing R³ represents a double bond. [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl; b) R¹ is C₁-C₃ alkyl; c) R⁵, R⁷, R⁸, R¹², R¹³ and R¹⁴ are independently selected from the group consisting of hydrogen, hydroxyl, mercaptan, keto, and C₁-C₃ alkyl; d) R⁶ is selected from the group consisting of hydrogen, hydroxyl, mercaptan, and C₁-C₃ alkyl; c) R⁹ is selected from the group consisting of hydrogen and —C(═O)R^{9a}; f) R¹⁰ is selected from the group consisting of hydrogen and —C(═O)R^{10a}; g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and h) R¹¹ is selected from the group consisting of hydrogen and C₁-C₃ alkyl. In some embodiments, R¹¹ is hydrogen or CH₃. In some embodiments, R¹¹ is CH₃. In some embodiments, R¹¹ is hydrogen. In some embodiments, R^{9a} is —CH₂CH₃ and/or R^{10a} are —CH₂CH₃. In some embodiments, the method comprises administering into an individual with anordrin or an analog thereof.

In some embodiments, the present application provides a composition and a method of using the composition for treating insomnia in an individual, wherein the composition comprises a compound of formula (I):

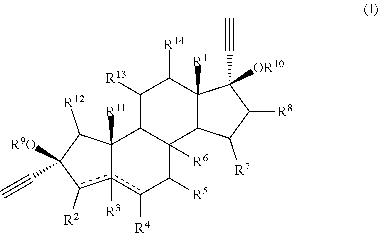

or a pharmaceutically acceptable salt or ester thereof, wherein: a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or [symbol] between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl; b) R¹ is C₁-C₃ alkyl; c) R⁵, R⁷, R⁸, R¹², R¹³ and R¹⁴ are independently selected from the group consisting of hydrogen, hydroxyl, mercaptan, keto, and C₁-C₃ alkyl; d) R⁶ is selected from the group consisting of hydrogen, hydroxyl, mercaptan, and C₁-C₃ alkyl; e) R⁹ is selected from the group consisting of hydrogen and —C(═O)R^{9a}; f) R¹⁰ is selected from the group consisting of hydrogen and —C(═O)R^{10a}; g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and h) R¹¹ is selected from the group consisting of C₁-C₃ alkyl. In some embodiments, R¹¹ is CH₃. In some embodiments, R^{9a} is —CH₂CH₃ and/or R^{10a} are —CH₂CH₃. In some embodiments, the method comprises administering into an individual with anordrin or an analog thereof.

The present application is at least partly based upon strikingly advantageous effects in treating insomnia exhibited by postmenopausal women under treatment of a representative compound, anordrin. Anordrin is one of selective estrogen receptor modulators (SERM), which are a class of synthetic non-steroidal agents that have varying estrogen agonist and antagonist activities in different tissues. How different SERMs act as an estrogen agonist in some tissues and estrogen antagonist in other tissues is not clearly established to this date. Most of the various SERMs that have been intensively tested do not have any known effects in treating insomnia. See Pinkerton et al. J Steroid Biochem Mol Biol. 2014 July; 142:142-54. In this application, it was demonstrated that 100% of the postmenopausal women having insomnia treated with anordrin at various dosages exhibited a significant improvement in sleeping. See Example 1. Moreover, anordrin has a favorable safety profile and can be used for long term. For at least these reasons, the compositions and methods described herein provide an ideal solution for women who have low estrogen level and suffer from insomnia.

### Definitions

The term “effective amount” used herein refers to an amount of a compound or composition sufficient to treat a specified disorder, condition or disease such as ameliorate, palliate, lessen, and/or delay one or more of its symptoms. In reference to insomnia, in some embodiments, an effective amount comprises an amount sufficient to cause a decrease in: 1) Athens Insomnia Scale (AIS) score (such as a decrease in self-assessed AIS score after treatment compared to before treatment); 2) Pittsburgh sleep quality index (PSQI) score (such as a decrease in self-assessed PSQI score after treatment compared to before treatment); 3) the extent of insomnia reflected by other sleep disorder questionnaire (SDQ) (such as Epworth Sleeping Scale); 4) sleep dysfunction rating scale (SDRS) by sleeping monitor; 5) or to prevent insomnia.

The term “individual” is a mammal, including humans. An individual includes, but is not limited to, human, bovine, horse, feline, canine, rodent, or primate. In some embodiments, the individual is human.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Alkyl” is a linear or branched saturated hydrocarbon. For example, an alkyl group can have 1 to 12 carbon atoms (i.e., (C₁-C₁₂alkyl)), or 1 to 10 carbon atoms (i.e., (C₁-C₁₀alkyl)), or 1 to 8 carbon atoms (i.e., (C₁-C₈alkyl)), or 1 to 6 carbon atoms (i.e., (C₁-C₆alkyl)), or 1 to 4 carbon atoms (i.e., (C₁-C₄alkyl)). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, isopropyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (1-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Cₓ alkyl refers to an alkyl group having x number of carbon atoms.

“Alkenyl” is a linear or branched hydrocarbon with at least one carbon-carbon double bond. For example, an alkenyl group can have 2 to 12 carbon atoms (i.e., C₂-C₁₂alkenyl), or 2 to 10 carbon atoms (i.e., C₂-C₁₀alkenyl), or 2 to 8 carbon atoms (i.e., C₂-C₈alkenyl), or 2 to 6 carbon atoms (i.e., C₂-C₆alkenyl), or 2 to 4 carbon atoms (i.e., C₂-C₄alkenyl). Examples of suitable alkenyl groups include, but are not limited to, ethylene or vinyl (—CH—CH₂), allyl (—CH₂CH═CH₂) and 5-hexenyl (—CH₂CH₂CH₂CH₂CH═CH₂). Cₓ alkenyl refers to an alkenyl group having x number of carbon atoms.

“Hydroxyl” refers to the moiety —OH.

“Keto” refers to the moiety (═O).

“Mercaptan” refers to the moiety —SH.

The terms “optionally substituted” as used throughout the specification means that a group may be unsubstituted or substituted by one or more (e.g., 1, 2, or 3) of the substituents listed for that group. For example, “C₁-C₆ alkyl optionally substituted with —C(═O)OH” means that the C₁-C₆ alkyl may but need not be substituted with —C(═O)OH. In one embodiment, an optionally substituted group has one substituent. In one embodiment, an optionally substituted group has two substituents. In one embodiment, an optionally substituted group is unsubstituted.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Methods of the Present Application

The present application provides methods of treating insomnia (e.g., chronic insomnia) in an individual, comprising administering to the individual an effective amount of a composition comprising a compound of formula A (e.g., anordrin) as described herein. The present application provides methods of treating insomnia (e.g., chronic insomnia) in an individual, comprising administering to the individual an effective amount of a composition comprising a compound of formula I (e.g., anordrin) as described herein. In some embodiments, R^{9a} is —CH₂CH₃ and/or R^{10a} are —CH₂CH₃. In some embodiments, the compound is anordrin. In some embodiments, the individual reaches menopause, is post menopause, or has been subject to a surgical removal of ovary. In some embodiments, the dose of the compound for each administration is about 20 μg to about 1 mg (such as about 20 μg to about 200 μg or about 400 μg to about 1 mg) for a human or a comparable dose for an individual that is not a human. In some embodiments, the compound is administered for at least about two weeks. In some embodiments, the compound is administered daily. In some embodiments, the compound is administered weekly.

The present application in one aspect provides a method of treating insomnia (e.g., chronic insomnia) in an individual, comprising administering to the individual an effective amount of a composition comprising a compound of formula A:

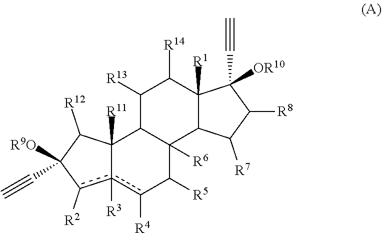

or a pharmaceutically acceptable salt or ester thereof, wherein: a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or [symbol] between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl; b) R¹ is C₁-C₃ alkyl; c) R⁵, R⁷, R⁸, R¹², R¹³ and R¹⁴ are independently selected from the group consisting of hydrogen, hydroxyl, mercaptan, keto, and C₁-C₃ alkyl; d) R⁶ is selected from the group consisting of hydrogen, hydroxyl, mercaptan, and C₁-C₃ alkyl; c) R⁹ is selected from the group consisting of hydrogen and —C(═O)R^{9a}; f) R¹⁰ is selected from the group consisting of hydrogen and —C(═O)R^{10a}; g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and h) R¹¹ is selected from the group consisting of hydrogen and C₁-C₃ alkyl. In some embodiments, R⁹ and R¹⁰ are independently hydrogen or —C(═O)CH₂CH₃. In some embodiments, R¹¹ is hydrogen. In some embodiments, R¹¹ is C₁-C₃ alkyl. In some embodiments, one or both of R¹ and R¹¹ are —CH₃. In some embodiments, R¹ is —CH₃ and R¹¹ is hydrogen. In some embodiments, both R¹ and R¹¹ are —CH₃. In some embodiments, any one or more of R², R³, R⁴, R⁵, R⁶, R⁷, R⁸, R¹¹, R¹², R¹³, and R¹⁴ are hydrogen. In some embodiments, any one or more of R², R³, R⁴, R⁵, R⁶, R⁷, R⁸, R¹², R¹³, and R¹⁴ are hydrogen. In some embodiments, R⁹ and R¹⁰ are independently hydrogen or —C(═O)CH₂CH₃. In some embodiments, any one or more (such as two, three, four, five, six, seven, eight, nine, ten, or eleven) of R², R³, R⁴, R⁵, R⁶, R⁷, R⁸, R¹¹, R¹², R¹³, and R¹⁴ are hydrogen. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, R^{9a} is —CH₂CH₃. In some embodiments, R^{10a} is —CH₂CH₃. In some embodiments, the compound is anordrin. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has a low level of estrogen. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed by other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the dose of the compound for each administration is at least about 20 μg (such as at least about 50 μg, 100 μg or 150 μg) for a human or a comparable dose for an individual that is not a human. In some embodiments, the dose of the compound for each administration is no more than 2 mg for a human or a comparable dose for an individual that is not a human. In some embodiments, the dose of the compound for each administration is about 20 μg to about 2 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the composition is administered orally.

The present application in one aspect provides a method of treating insomnia (e.g., chronic insomnia) in an individual, comprising administering to the individual an effective amount of a composition comprising a compound of formula I:

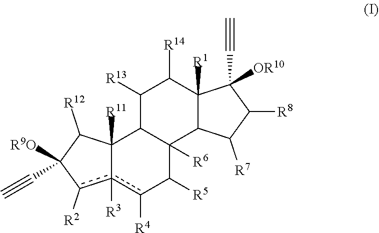

or a pharmaceutically acceptable salt or ester thereof, wherein: a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or [symbol] between the carbon bearing R² and the carbon bearing R³ represents a double bond, [symbol] between the carbon bearing R³ and the carbon bearing R⁴ represents a single bond. R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl; b) R¹ is C₁-C₃ alkyl; c) R⁵, R⁷, R⁸, R¹², R¹³ and R¹⁴ are independently selected from the group consisting of hydrogen, hydroxyl, mercaptan, keto, and C₁-C₃ alkyl; d) R⁶ is selected from the group consisting of hydrogen, hydroxyl, mercaptan, and C₁-C₃ alkyl; c) R⁹ is selected from the group consisting of hydrogen and —C(═O)R^{9a}; f) R¹⁰ is selected from the group consisting of hydrogen and —C(═O)R^{10a}; g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and h) R¹¹ is selected from the group consisting of C₁-C₃ alkyl. In some embodiments, R⁹ and R¹⁰ are independently hydrogen or —C(═O)CH₂CH₃. In some embodiments, one or both of R¹ and R¹¹ are —CH₃. In some embodiments, any one or more of R², R³, R⁴, R⁵, R⁶, R⁷, R⁸, R¹², R¹³, and R¹⁴ are hydrogen. In some embodiments, R⁹ and R¹⁰ are independently hydrogen or —C(═O)CH₂CH₃. In some embodiments, one or both of R¹ and R¹¹ are —CH₃. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, R^{9a} is —CH₂CH₃. In some embodiments, R^{10a} is —CH₂CH₃. In some embodiments, the compound is anordrin. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has a low level of estrogen. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed by other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the dose of the compound for each administration is at least about 20 μg (such as at least about 50 μg, 100 μg or 150 μg) for a human or a comparable dose for an individual that is not a human. In some embodiments, the dose of the compound for each administration is no more than 2 mg for a human or a comparable dose for an individual that is not a human. In some embodiments, the dose of the compound for each administration is about 20 μg to about 2 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered intravenously, intraarterially, intraperitoneally, intravesicularly, subcutaneously, intrathecally, intrapulmonarily, intramuscularly, intratrachcally, intraocularly, transdermally, orally, or by inhalation. In some embodiments, the composition is administered orally.

In some embodiments, there is provided a method of treating insomnia (e.g., chronic insomnia) in an individual, comprising administering to the individual an effective amount of a composition comprising a compound of formula II-A, III-A or IV-A, or a pharmaceutically acceptable salt or ester thereof, wherein R⁹ is selected from the group consisting of hydrogen and —C(═O)R^{9a}; R¹⁰ is selected from the group consisting of hydrogen and —C(═O)R^{10a}; R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; R′ is selected from the group consisting of hydrogen and C₁-C₃ alkyl; and R¹¹ is selected from the group consisting of C₁-C₃ alkyl.

In some embodiments, the composition comprises a compound of formula II-A-1, III-A-1, or IV-A-1 or a pharmaceutically acceptable salt or ester thereof. In some embodiments, the composition comprises a compound of formula II-A-1-a, III-A-1-a, or IV-A-1-a. In some embodiments, R⁹ and R¹⁰ are independently hydrogen or —C(═O)CH₂CH₃. In some embodiments, R^{9a} is —CH₂CH₃. In some embodiments, R^{10a} is —CH₂CH₃. In some embodiments, one or both of R¹ and R¹¹ are —CH₃. In some embodiments, the compound is anordrin. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has an aberrant level of hormone (such as estrogen). In some embodiments, the individual has a score of at least about 7 according to Athens Insomnia Scale. In some embodiments, the individual has a score of at least about 11 according to Pittsburgh sleep quality index (PSQI). In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with an aberrant level of estrogen. In some embodiments, insomnia is associated with perimenopause, menopause, or postmenopause. In some embodiments, the dose of the compound for each administration is about 20 μg to about 2 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

In some embodiments, there is provided a method of treating insomnia (e.g., chronic insomnia) in an individual, comprising administering to the individual an effective amount of a composition comprising a compound of any one of formula II-B, III-B, IV-B, II-C, III-C, and IV-C, or a pharmaceutically acceptable salt or ester thereof, wherein a) R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl or absent; b) R¹ is C₁-C₃ alkyl; c) R⁵, R⁷, R⁸, R¹², R¹³ and R¹⁴ are independently selected from the group consisting of hydrogen, hydroxyl, mercaptan, keto, and C₁-C₃ alkyl; d) R⁶ is selected from the group consisting of hydrogen, hydroxyl, mercaptan, and C₁-C₃ alkyl; c) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and f) R¹¹ is selected from the group consisting of C₁-C₃ alkyl. In some embodiments, the composition comprises a compound of formula II-B, III-B, or IV-B or a pharmaceutically acceptable salt or ester thereof. In some embodiments, the composition comprises a compound of formula II-C, III-C, or IV-C or a pharmaceutically acceptable salt or ester thereof. In some embodiments, the composition comprises a compound of formula II-C-1, III-C-1, or IV-C-1 or a pharmaceutically acceptable salt or ester thereof. In some embodiments, R¹ and R¹¹ are independently C₁ alkyl. In some embodiments, R⁹ and R¹⁰ are independently hydrogen or —C(═O)CH₂CH₃. In some embodiments, R^{9a} is —CH₂CH₃. In some embodiments, R^{10a} is —CH₂CH₃. In some embodiments, one or both of R¹ and R¹¹ are —CH₃. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, the compound is anordrin. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has a low level of estrogen. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the dose of the compound for each administration is about 20 μg to about 2 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

In some embodiments, there is provided a method of treating insomnia (e.g., chronic insomnia) in an individual, comprising administering to the individual an effective amount of a composition comprising a compound of formula A (e.g., anordrin) as described herein or a pharmaceutically acceptable salt or ester thereof, wherein the individual is at perimenopause stage, reaches menopause, is post menopause, or has been subject to a surgical removal of ovary. In some embodiments, there is provided a method of treating insomnia (e.g., chronic insomnia) in an individual, comprising administering to the individual an effective amount of a composition comprising a compound of formula I (e.g., anordrin) as described herein or a pharmaceutically acceptable salt or ester thereof, wherein the individual is at perimenopause stage, reaches menopause, is post menopause, or has been subject to a surgical removal of ovary. In some embodiments, the individual is selected for treatment based upon the individual being at perimenopause stage, reaches menopause, is post menopause, or has been subject to a surgical removal of ovary. In some embodiments, the individual is a postmenopausal woman. In some embodiments, the composition comprises a compound of formula II-A, III-A, or IV-A (such as II-A-1, III-A-1, or IV-A-1, such as II-A-1-a, III-A-1-a, or IV-A-1-a) or a pharmaceutically acceptable salt or ester thereof. In some embodiments, the composition comprises a compound of any one of formula II-B, III-B, IV-B, II-C, III-C, and IV-C, or a pharmaceutically acceptable salt or ester thereof. In some embodiments, the composition comprises a compound of formula II-B, III-B, or IV-B or a pharmaceutically acceptable salt or ester thereof. In some embodiments, the composition comprises a compound of formula II-C, III-C, or IV-C or a pharmaceutically acceptable salt or ester thereof. In some embodiments, the composition comprises a compound of formula II-C-1, III-C-1, or IV-C-1 or a pharmaceutically acceptable salt or ester thereof. In some embodiments, R⁹ and R¹⁰ are independently hydrogen or —C(═O)CH₂CH₃. In some embodiments, R^{9a} is —CH₂CH₃. In some embodiments, R^{10a} is —CH₂CH₃. In some embodiments, R¹¹ is hydrogen. In some embodiments, R¹¹ is C₁-C₃ alkyl. In some embodiments, one or both of R¹ and R¹¹ are —CH₃. In some embodiments, R¹ is —CH₃ and R¹¹ is hydrogen. In some embodiments, both R¹ and R¹¹ are —CH₃. In some embodiments, any one or more (such as two, three, four, five, six, seven, eight, nine, ten, or eleven) of R², R³, R⁴, R⁵, R⁶, R⁷, R⁸, R¹¹, R¹², R¹³, and R¹⁴ are hydrogen. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, the compound is anordrin. In some embodiments, the individual is a human. In some embodiments, the individual has a low level of estrogen. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the dose of the compound for each administration is about 20 μg to about 1 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

In some embodiments, there is provided a method of treating insomnia (e.g., chronic insomnia) in an individual, comprising administering to the individual an effective amount of a composition comprising a compound of formula A (e.g., anordrin) as described herein or a pharmaceutically acceptable salt or ester thereof, wherein the individual has an low level of hormone (e.g., estrogen). In some embodiments, there is provided a method of treating insomnia (e.g., chronic insomnia) in an individual, comprising administering to the individual an effective amount of a composition comprising a compound of formula I (e.g., anordrin) as described herein or a pharmaceutically acceptable salt or ester thereof, wherein the individual has an low level of hormone (e.g., estrogen). In some embodiments, the individual has a serum estrogen level (e.g., serum estradiol (E2) level, e.g., an average serum estradiol level) of less than 35 pg/mL or 18 pg/mL. In some embodiments, the individual has a scrum FSH level higher than 40 IU/L. In some embodiments, the composition comprises a compound of formula II-A, III-A, or IV-A (such as II-A-1, III-A-1, or IV-A-1, such as II-A-1-a, III-A-1-a, or IV-A-1-a) or a pharmaceutically acceptable salt or ester thereof. In some embodiments, the composition comprises a compound of any one of formula II-B, III-B, IV-B, II-C, III-C, and IV-C, or a pharmaceutically acceptable salt or ester thereof. In some embodiments, the composition comprises a compound of formula II-B, III-B, or IV-B or a pharmaceutically acceptable salt or ester thereof. In some embodiments, the composition comprises a compound of formula II-C, III-C, or IV-C or a pharmaceutically acceptable salt or ester thereof. In some embodiments, the composition comprises a compound of formula II-C-1, III-C-1, or IV-C-1 or a pharmaceutically acceptable salt or ester thereof. In some embodiments, R⁹ and R¹⁰ are independently hydrogen or —C(═O)CH₂CH₃. In some embodiments, R^{9a} is —CH₂CH₃. In some embodiments, R^{10a} is —CH₂CH₃. In some embodiments, R¹¹ is hydrogen. In some embodiments, R¹¹ is C₁-C₃ alkyl. In some embodiments, one or both of R¹ and R¹¹ are —CH₃. In some embodiments, R¹ is —CH₃ and R¹¹ is hydrogen. In some embodiments, both R¹ and R¹¹ are —CH₃. In some embodiments, any one or more (such as two, three, four, five, six, seven, eight, nine, ten, or eleven) of R², R³, R⁴, R⁵, R⁶, R⁷, R⁸, R¹¹, R¹², R¹³, and R¹⁴ are hydrogen. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, the compound is anordrin. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with an aberrant level of estrogen. In some embodiments, insomnia is associated with perimenopause, menopause, or postmenopause. In some embodiments, the dose of the compound for each administration is about 20 μg to about 1 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

In some embodiments, there is provided a method of treating insomnia (e.g., chronic insomnia) in a human individual, comprising administering to the human individual an effective amount of a composition comprising a compound of formula A (e.g., anordrin) as described herein or a pharmaceutically acceptable salt or ester thereof, wherein the dose of the compound for each administration is about 20 μg to about 2 mg (such as about 20 μg to about 200 μg or about 400 μg to about 2 mg) for a human. In some embodiments, there is provided a method of treating insomnia (e.g., chronic insomnia) in a human individual, comprising administering to the human individual an effective amount of a composition comprising a compound of formula I (e.g., anordrin) as described herein or a pharmaceutically acceptable salt or ester thereof, wherein the dose of the compound for each administration is about 20 μg to about 2 mg (such as about 20 μg to about 200 μg or about 400 μg to about 2 mg) for a human. In some embodiments, the composition is administered daily. In some embodiments, the composition is administered daily at a dosage of about 20 μg to about 200 μg (such as about 50 μg, about 100 μg, or about 150 μg). In some embodiments, the composition is administered weekly. In some embodiments, the composition is administered weekly at a dosage of about 400 μg to about 2 mg. In some embodiments, the composition comprises a compound of formula II-A, III-A, or IV-A (such as II-A-1, III-A-1, or IV-A-1, such as II-A-1-a, III-A-1-a, or IV-A-1-a) or a pharmaceutically acceptable salt or ester thereof. In some embodiments, the composition comprises a compound of any one of formula II-B, III-B, IV-B, II-C, III-C, and IV-C, or a pharmaceutically acceptable salt or ester thereof. In some embodiments, the composition comprises a compound of formula II-B, III-B, or IV-B or a pharmaceutically acceptable salt or ester thereof. In some embodiments, the composition comprises a compound of formula II-C, III-C, or IV-C or a pharmaceutically acceptable salt or ester thereof. In some embodiments, the composition comprises a compound of formula II-C-1, III-C-1, or IV-C-1 or a pharmaceutically acceptable salt or ester thereof. In some embodiments, R⁹ and R¹⁰ are independently hydrogen or —C(═O)CH₂CH₃. In some embodiments, R^{9a} is —CH₂CH₃. In some embodiments, R^{10a} is —CH₂CH₃. In some embodiments, R¹¹ is hydrogen. In some embodiments, R¹¹ is C₁-C₃ alkyl. In some embodiments, one or both of R¹ and R¹¹ are —CH₃. In some embodiments, R¹ is —CH₃ and R¹¹ is hydrogen. In some embodiments, both R¹ and R¹¹ are —CH₃. In some embodiments, any one or more (such as two, three, four, five, six, seven, eight, nine, ten, or eleven) of R², R³, R⁴, R⁵, R⁶, R⁷, R⁸, R¹¹, R¹², R¹³, and R¹⁴ are hydrogen. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, the compound is anordrin. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has a low level of hormone (such as estrogen). In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the dose of the compound for each administration is about 20 μg to about 2 mg (such as about 20 μg to about 200 μg or about 400 μg to about 2 mg) for a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

In some embodiments, there is provided a method of treating insomnia (e.g., chronic insomnia) in a female individual, comprising administering to the individual an effective amount of a composition comprising a compound of any one of formula selected from the group consisting of II-A-1-a, III-A-1-a, IV-A-1-a, II-C, III-C, and IV-C, or a pharmaceutically acceptable salt or ester thereof, wherein both of R¹ and R¹¹ are —CH₃, wherein the individual a) is at perimenopause stage, reaches menopause, is post menopause, or has been subject to a surgical removal of ovary or b) has an aberrant level of hormone (e.g., estrogen). In some embodiments, R^{9a} is —CH₂CH₃. In some embodiments, R^{10a} is —CH₂CH₃. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, the compound is anordrin. In some embodiments, the individual is a human. In some embodiments, the individual is a postmenopausal woman. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the dose of the compound for each administration is about 20 μg to about 2 mg (such as about 20 μg to about 200 μg or about 400 μg to about 2 mg) for a human or a comparable dose for an individual that is not a human. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 1 mg. In some embodiments, the composition is administered orally.

In some embodiments, there is provided a method of treating insomnia (e.g., chronic insomnia) in an individual, comprising administering to the individual an effective amount of a composition comprising a compound of any one of formula selected from the group consisting of II-A-1-a, III-A-1-a, IV-A-1-a, II-C, III-C, and IV-C, or a pharmaceutically acceptable salt or ester thereof, wherein both of R¹ and R¹¹ are —CH₃, wherein the dose of the compound for each administration is about 20 μg to about 1 mg (such as about 20 μg to about 200 μg or about 400 μg to about 2 mg) for a human. In some embodiments, R^{9a} is —CH₂CH₃. In some embodiments, R^{10a} is —CH₂CH₃. In some embodiments, any one or more (such as two, three, four, five, six, seven, eight, nine, or ten) of R², R³, R⁴, R⁵, R⁶, R⁷, R⁸, R¹², R¹³, and R¹⁴ are hydrogen. In some embodiments, the compound is anordrin. In some embodiments, the composition is administered daily. In some embodiments, the composition is administered daily at a dosage of about 20 μg to about 200 μg (such as about 50 μg, about 100 μg, or about 150 μg). In some embodiments, the composition is administered weekly. In some embodiments, the composition is administered weekly at a dosage of about 400 μg to about 2 mg. In some embodiments, the individual is a female. In some embodiments, the individual is a human. In some embodiments, the individual reaches menopause, is post menopause, or has been subject to a surgical removal of ovary. In some embodiments, the individual is a postmenopausal woman. In some embodiments, the individual has a low level of hormone (such as estrogen). In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

In some embodiments, there is provided a method of treating insomnia (e.g., chronic insomnia) in a female human individual, comprising administering to the individual an effective amount of a composition comprising anordrin or an analog thereof (such as anordrin), wherein the individual a) is at perimenopause stage, reaches menopause, is post menopause, or has been subject to a surgical removal of ovary or b) has a low level of estrogen, and optionally wherein the dose of anordrin or the analog thereof for each administration is about 20 μg to about 2 mg (such as about 20 μg to about 200 μg or about 400 μg to about 2 mg) for a human or a comparable dose for an individual that is not a human. In some embodiments, the composition is administered daily at a dosage of about 20 μg to about 200 μg (such as about 50 μg, about 100 μg, or about 150 μg). In some embodiments, the composition is administered weekly. In some embodiments, the composition is administered weekly at a dosage of about 400 μg to about 2 mg. In some embodiments, the individual is a postmenopausal woman. In some embodiments, the individual has a score of at least about 7 according to Athens Insomnia Scale. In some embodiments, the individual has a score of at least about 11 according to Pittsburgh sleep quality index. PSQI. In some embodiments, the insomnia is assessed with other sleep disorder questionnaire (SDQ) such as Epworth Sleeping Scale or sleep dysfunction rating scale (SDRS) by sleeping monitor. In some embodiments, the individual is not subject or has not been subject to a hormone therapy. In some embodiments, insomnia is associated with a low level of estrogen. In some embodiments, insomnia is associated with perimenopause, menopause, or postmenopause. In some embodiments, the composition is administered at a frequency of about once every two weeks to about three times a day. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg. In some embodiments, the composition is administered orally.

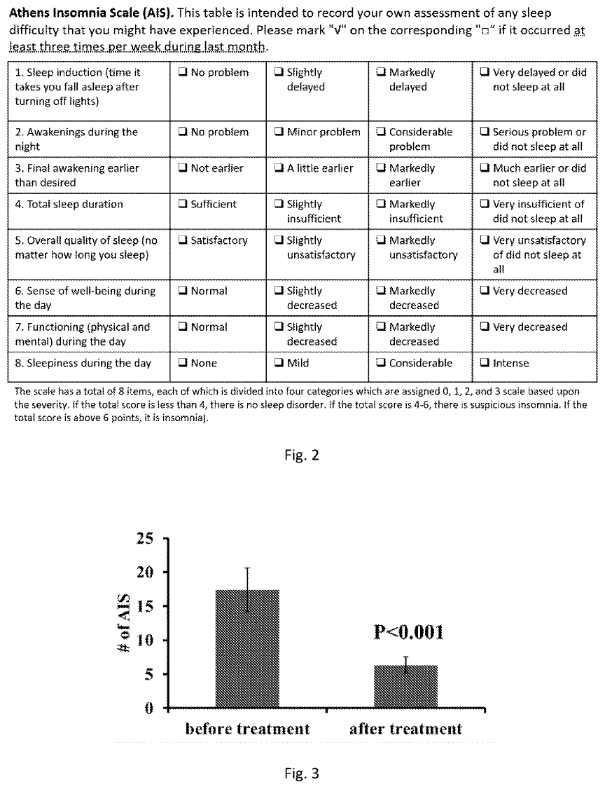

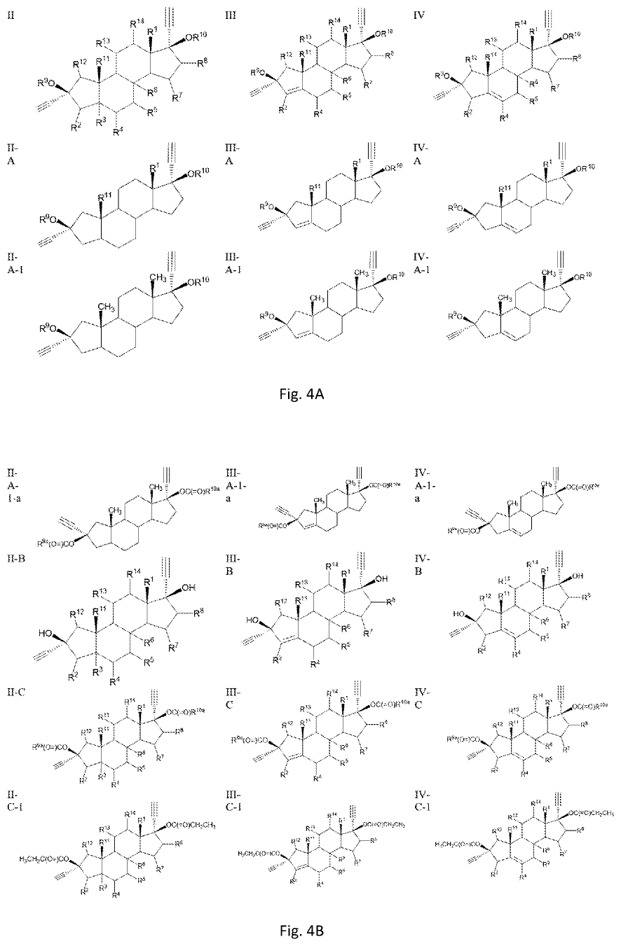

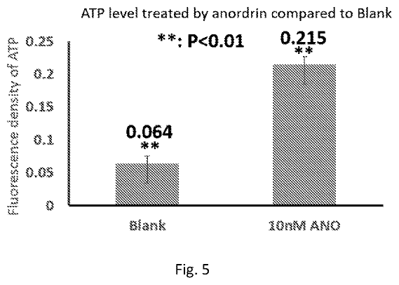

In some embodiments, there is provided a method of improving sleeping in an individual, comprising administering (e.g., orally administering) to the individual an effective amount of a composition comprising a compound of formula A (e.g., anordrin) as described herein or a pharmaceutically acceptable salt or ester thereof. In some embodiments, there is provided a method of improving sleeping in an individual, comprising administering (e.g., orally administering) to the individual an effective amount of a composition comprising a compound of formula I (e.g., anordrin) as described herein or a pharmaceutically acceptable salt or ester thereof. In some embodiments, the individual is a female individual. In some embodiments, the individual is at perimenopause stage, reaches menopause, is post menopause, or has been subject to a surgical removal of ovary. In some embodiments, the individual has a low level of hormone (e.g., estrogen). In some embodiments, improving sleeping comprises improving any one or more (such as two, three, four, five, six, seven, or eight of the events evaluated under Athens Insomnia Scale (see FIG. 2). For example, the events include sleep induction time, awakening during the night, final awakening earlier than desired, total sleep duration, overall quality of sleep, sense of well-being during the day, functioning, sleepiness during the day. In some embodiments, the improvement of these events can be indicated by the individual's self-assessment before and after the administration of compound. In some embodiments, the self-assessment after the administration is conducted after the administration of the compound at a frequency and/or dose described herein for at least about one, two, three, four, five or six weeks.

In some embodiments, there is provided a method of alleviating severity of insomnia in an individual, comprising administering (e.g., orally administering) to the individual an effective amount of a composition comprising a compound of formula A (e.g., anordrin) as described herein or a pharmaceutically acceptable salt or ester thereof. In some embodiments, there is provided a method of alleviating severity of insomnia in an individual, comprising administering (e.g., orally administering) to the individual an effective amount of a composition comprising a compound of formula I (e.g., anordrin) as described herein or a pharmaceutically acceptable salt or ester thereof. In some embodiments, the insomnia score is decreased by at least about 10%. 20%. 30%, 40%, 50%, or 60% after treatment at a frequency and dosage for a duration of time as described herein. In some embodiments, the individual is a female individual. In some embodiments, the individual is at perimenopause stage, reaches menopause, is post menopause, or has been subject to a surgical removal of ovary. In some embodiments, the individual has a low level of hormone (e.g., estrogen). In some embodiments, improving sleeping comprises improving any one or more (such as two, three, four, five, six, seven, or eight of the events evaluated under Athens Insomnia Scale (see FIG. 2). For example, the events include sleep induction time, awakening during the night, final awakening earlier than desired, total sleep duration, overall quality of sleep, sense of well-being during the day, functioning, and sleepiness during the day. In some embodiments, the improvement of these events can be indicated by the individual's self-assessment before and after the administration of compound. In some embodiments, the self-assessment after the administration is conducted after the administration of the compound at a frequency and/or dose described herein for at least about one, two, three, four, five or six weeks.

In some embodiments, there is provided a method of increasing duration of sleep in an individual, comprising administering (e.g., orally administering) to the individual an effective amount of a composition comprising a compound of formula A (e.g., anordrin) as described herein or a pharmaceutically acceptable salt or ester thereof. In some embodiments, there is provided a method of shortening sleep induction time for an individual, comprising administering (e.g., orally administering) to the individual an effective amount of a composition comprising a compound of formula A (e.g., anordrin) as described herein or a pharmaceutically acceptable salt or ester thereof. In some embodiments, there is provided a method of decreasing awakening times during night for an individual, comprising administering (e.g., orally administering) to the individual an effective amount of a composition comprising a compound of formula A (e.g., anordrin) as described herein or a pharmaceutically acceptable salt or ester thereof. In some embodiments, there is provided a method of improving overall quality of sleep in an individual, comprising administering (e.g., orally administering) to the individual an effective amount of a composition comprising a compound of formula A (e.g., anordrin) as described herein or a pharmaceutically acceptable salt or ester thereof. In some embodiments, there is provided a method of improving quality of life for an individual, comprising administering (e.g., orally administering) to the individual an effective amount of a composition comprising a compound of formula A (e.g., anordrin) as described herein or a pharmaceutically acceptable salt or ester thereof.

In some embodiments, there is provided a method of increasing duration of sleep in an individual, comprising administering (e.g., orally administering) to the individual an effective amount of a composition comprising a compound of formula I (e.g., anordrin) as described herein or a pharmaceutically acceptable salt or ester thereof. In some embodiments, there is provided a method of shortening sleep induction time for an individual, comprising administering (e.g., orally administering) to the individual an effective amount of a composition comprising a compound of formula I (e.g., anordrin) as described herein or a pharmaceutically acceptable salt or ester thereof. In some embodiments, there is provided a method of decreasing awakening times during night for an individual, comprising administering (e.g., orally administering) to the individual an effective amount of a composition comprising a compound of formula I (e.g., anordrin) as described herein or a pharmaceutically acceptable salt or ester thereof. In some embodiments, there is provided a method of improving overall quality of sleep in an individual, comprising administering (e.g., orally administering) to the individual an effective amount of a composition comprising a compound of formula I (e.g., anordrin) as described herein or a pharmaceutically acceptable salt or ester thereof. In some embodiments, there is provided a method of improving quality of life for an individual, comprising administering (e.g., orally administering) to the individual an effective amount of a composition comprising a compound of formula I (e.g., anordrin) as described herein or a pharmaceutically acceptable salt or ester thereof.

In some embodiments, there is provided a method of treating insomnia (e.g., chronic insomnia) or improving sleeping in an individual, comprising administering to the individual an effective amount of a composition comprising a compound of formula A (e.g., anordrin) as described herein or a pharmaceutically acceptable salt or ester thereof for at least about two weeks (such as at least about three, four, five, six, seven, or eight weeks). In some embodiments, there is provided a method of treating insomnia (e.g., chronic insomnia) or improving sleeping in an individual, comprising administering to the individual an effective amount of a composition comprising a compound of formula I (e.g., anordrin) as described herein or a pharmaceutically acceptable salt or ester thereof for at least about two weeks (such as at least about three, four, five, six, seven, or eight weeks). In some embodiments, the composition is administered for at least about two, three, four, five, six, seven, eight, nine, ten, eleven, twelve, fifteen, or eighteen months. In some embodiments, the composition is administered for at least about one year, one and a half years, two years, three years, four years, or five years. In some embodiments, the composition is administered daily. In some embodiments, the composition is administered daily at a dosage of about 20 μg to about 200 μg. In some embodiments, the composition is administered weekly. In some embodiments, the composition is administered weekly at a dosage of about 400 μg to about 2 mg.

In some embodiments according to any of the methods described above, the individual has a low level of estrogen. In some embodiments, the low level of hormone (e.g., estrogen) is a hormone (e.g., estrogen) level that is lower than a control level (such as at least about any of 18 pg/mL, or 35 pg/mL). In some embodiments, the individual has a serum FSH level higher than about 20, 30, or 40 IU/L. In some embodiments, the individual has a serum estrogen level (e.g., serum estradiol (E2) level, e.g., an average serum estradiol level) of less than about 35 pg/mL or 18 pg/mL. In some embodiments, the individual has endometrium that has a thickness of no more than about 7 mm. 6 mm. 5 mm. 4.5 mm, or 4 mm.

In some embodiments, the method further comprises administering a second agent.

In some embodiments, the method further comprises assessing hormone (e.g., estrogen) level in the individual. In some embodiments, the method further comprises selecting an individual for treatment based upon hormone level (e.g., serum estrogen level). Methods of assessing estrogen level are well known in the field. For example, serum estrogen level can be assessed by measuring estradiol (E2) in blood.

### Compositions and Compounds

The present application in one aspect provides compositions and compounds that can be used for treating insomnia in methods described herein.

In one aspect, provided herein is a composition comprising a compound of formula (A):

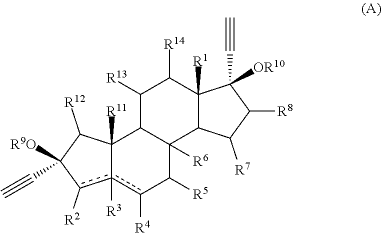

or a pharmaceutically acceptable salt or ester thereof, wherein:
   a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or
      [symbol] between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or
      [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl;
   b) R¹ is C₁-C₃ alkyl;
   c) R⁵, R⁷, R⁸, R¹², R¹³ and R¹⁴ are independently selected from the group consisting of hydrogen, hydroxyl, mercaptan, keto, and C₁-C₃ alkyl;
   d) R⁶ is selected from the group consisting of hydrogen, hydroxyl, mercaptan, and C₁-C₃ alkyl;
   e) R⁹ is selected from the group consisting of hydrogen and —C(═O)R^{9a};
   f) R¹⁰ is selected from the group consisting of hydrogen and —C(═O)R^{10a};
   g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and
   h) R¹¹ is selected from the group consisting of hydrogen and C₁-C₃ alkyl.

In one aspect, provided herein is a composition comprising a compound of formula (I):

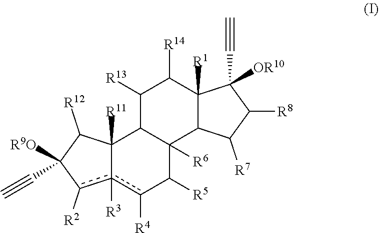

or a pharmaceutically acceptable salt or ester thereof, wherein:
   a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or
      [symbol] between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or
      [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl;
   b) R¹ is C₁-C₃ alkyl;
   c) R⁵, R⁷, R⁸, R¹², R¹³ and R¹⁴ are independently selected from the group consisting of hydrogen, hydroxyl, mercaptan, keto, and C₁-C₃ alkyl;
   d) R⁶ is selected from the group consisting of hydrogen, hydroxyl, mercaptan, and C₁-C₃ alkyl;
   e) R⁹ is selected from the group consisting of hydrogen and —C(═O)R^{9a};
   f) R¹⁰ is selected from the group consisting of hydrogen and —C(═O)R^{10a};
   g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and
   h) R¹¹ is selected from the group consisting of C₁-C₃ alkyl.

In some embodiments, [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl. In some embodiments, R², R³, and R⁴ are independently selected from the group consisting of hydrogen, hydroxyl, mercaptan, and C₁-C₃ alkyl (i.e., methyl, ethyl, n-propyl, or isopropyl). In some embodiments, R², R³, and R⁴ are independently selected from the group consisting of hydrogen, hydroxyl, and —CH₃. In some embodiments, R², R³, and R⁴ are independently selected from the group consisting of hydrogen, hydroxyl, and —CH₂CH₃. In some embodiments, R², R³, and R⁴ are each hydrogen. In some embodiments, one of R², R³, and R⁴ is hydroxyl and the others are each hydrogen. In some embodiments, R² is hydroxyl, and R³ and R⁴ are each hydrogen. In some embodiments, R³ is hydroxyl, and R² and R⁴ are each hydrogen. In some embodiments, R⁴ is hydroxyl, and R² and R³ are each hydrogen. In some embodiments, one of R², R³, and R⁴ is mercaptan and the others are each hydrogen. In some embodiments, R² is mercaptan, and R³ and R⁴ are each hydrogen. In some embodiments, R³ is mercaptan, and R² and R⁴ are each hydrogen. In some embodiments, R⁴ is mercaptan, and R² and R³ are each hydrogen. In some embodiments, one of R², R³, and R⁴ is —CH₃ and the others are each hydrogen. In some embodiments, one of R², R³, and R⁴ is —CH₂CH₃ and the others are each hydrogen. In some embodiments, one of R², R³, and R⁴ is C₃ alkyl (i.e., n-propyl or isopropyl) and the others are each hydrogen. In some embodiments, R² is C₁-C₃ alkyl (i.e., methyl, ethyl, n-propyl, or isopropyl), and R³ and R⁴ are each hydrogen. In some embodiments, R³ is C₁-C₃ alkyl (i.e., methyl, ethyl, n-propyl, or isopropyl), and R² and R⁴ are each hydrogen. In some embodiments, R⁴ is C₁-C₃ alkyl (i.e., methyl, ethyl, n-propyl, or isopropyl), and R² and R³ are each hydrogen. In some embodiments, one of R² and R⁴ is keto. In some embodiments, R² is keto, and R³ and R⁴ are selected from the group consisting of hydrogen, hydroxyl, mercaptan, and C₁-C₃ alkyl (i.e., methyl, ethyl, n-propyl, or isopropyl). In some embodiments, R² is keto, and R³ and R⁴ are each hydrogen. In some embodiments, R² is keto. R³ is hydrogen, and R⁴ is hydroxyl. In some embodiments, R² is keto. R³ is hydrogen, and R⁴ is mercaptan. In some embodiments, R² is keto. R³ is hydrogen, and R⁴ is C₁-C₃ alkyl. In some embodiments, R² is keto. R³ is hydrogen, and R⁴ is —CH₃. In some embodiments, R⁴ is keto, and R² and R³ are selected from the group consisting of hydrogen, hydroxyl, mercaptan, and C₁-C₃ alkyl (i.e., methyl, ethyl, n-propyl, or isopropyl). In some embodiments, R⁴ is keto, and R² and R³ are each hydrogen. In some embodiments, R⁴ is keto, R³ is hydrogen, and R² is hydroxyl. In some embodiments, R⁴ is keto. R³ is hydrogen, and R² is mercaptan. In some embodiments, R⁴ is keto, R³ is hydrogen, and R² is C₁-C₃ alkyl. In some embodiments, R⁴ is keto. R³ is hydrogen, and R² is —CH₃. In some embodiments, both of R² and R⁴ are keto. In some embodiments, both of R² and R⁴ are keto, and R³ is hydrogen.

In some embodiments, [symbol] between the carbon bearing R² and the carbon bearing R³ represents a single bond. [symbol] between the carbon bearing R³ and the carbon bearing R⁴ represents a single bond. R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl. In some embodiments, R³ is hydrogen. In some embodiments, R³ is hydroxyl. In some embodiments, R³ is mercaptan. In some embodiments, R³ is C₁-C₃ alkyl (i.e., methyl, ethyl, n-propyl, or isopropyl). In some embodiments, R³ is —CH₃.

In some embodiments, [symbol] between the carbon bearing R² and the carbon bearing R³ represents a double bond. [symbol] between the carbon bearing R³ and the carbon bearing R⁴ represents a single bond. R² is selected from the group consisting of hydrogen, hydroxyl, mercaptan, and C₁-C₃ alkyl. R³ is absent, and R⁴ is selected from the group consisting of hydrogen, hydroxyl, mercaptan, keto, and C₁-C₃ alkyl. In some embodiments, R² and R⁴ are independently selected from the group consisting of hydrogen, hydroxyl, mercaptan, and C₁-C₃ alkyl (i.e., methyl, ethyl, n-propyl, or isopropyl). In some embodiments, R² and R⁴ are independently selected from the group consisting of hydrogen, hydroxyl, and —CH₃. In some embodiments, R² and R⁴ are independently selected from the group consisting of hydrogen, hydroxyl, and —CH₂CH₃. In some embodiments, R² and R⁴ are each hydrogen. In some embodiments, R² is hydroxyl, and R⁴ is hydrogen. In some embodiments, R⁴ is hydroxyl, and R² is hydrogen. In some embodiments, R² is mercaptan, and R⁴ is hydrogen. In some embodiments, R⁴ is mercaptan, and R² is hydrogen. In some embodiments, R² is C₁-C₃ alkyl (i.e., methyl, ethyl, n-propyl, or isopropyl), and R⁴ is hydrogen. In some embodiments, R² is —CH₃, and R⁴ is hydrogen. In some embodiments, R⁴ is C₁-C₃ alkyl (i.e., methyl, ethyl, n-propyl, or isopropyl), and R² is hydrogen. In some embodiments, R⁴ is —CH₃, and R² is hydrogen. In some embodiments, R⁴ is keto, and R² is selected from the group consisting of hydrogen, hydroxyl, mercaptan, and C₁-C₃ alkyl. In some embodiments, R⁴ is keto, and R² is hydrogen. In some embodiments, R⁴ is keto, and R² is hydroxyl. In some embodiments, R⁴ is keto, and R² is mercaptan. In some embodiments, R⁴ is keto, and R² is C₁-C₃ alkyl. In some embodiments, R⁴ is keto, and R² is —CH₃.

In some embodiments, [symbol] between the carbon bearing R² and the carbon bearing R³ represents a single bond. [symbol] between the carbon bearing R³ and the carbon bearing R⁴ represents a double bond. R² is selected from the group consisting of hydrogen, hydroxyl, mercaptan, keto, and C₁-C₃ alkyl. R³ is absent, and R⁴ is selected from the group consisting of hydrogen, hydroxyl, mercaptan, and C₁-C₃ alkyl. In some embodiments, R² and R⁴ are independently selected from the group consisting of hydrogen, hydroxyl, mercaptan, and C₁-C₃ alkyl (i.e., methyl, ethyl, n-propyl, or isopropyl). In some embodiments, R² and R⁴ are independently selected from the group consisting of hydrogen, hydroxyl, and —CH₃. In some embodiments, R² and R⁴ are independently selected from the group consisting of hydrogen, hydroxyl, and —CH₂CH₃. In some embodiments, R² and R⁴ are each hydrogen. In some embodiments, R² is hydroxyl, and R⁴ is hydrogen. In some embodiments, R⁴ is hydroxyl, and R² is hydrogen. In some embodiments, R² is mercaptan, and R⁴ is hydrogen. In some embodiments, R⁴ is mercaptan, and R² is hydrogen. In some embodiments, R² is C₁-C₃ alkyl (i.e., methyl, ethyl, n-propyl, or isopropyl), and R⁴ is hydrogen. In some embodiments, R² is —CH₃, and R⁴ is hydrogen. In some embodiments, R⁴ is C₁-C₃ alkyl (i.e., methyl, ethyl, n-propyl, or isopropyl), and R² is hydrogen. In some embodiments, R⁴ is —CH₃, and R² is hydrogen. In some embodiments, R² is keto, and R⁴ is selected from the group consisting of hydrogen, hydroxyl, mercaptan, and C₁-C₃ alkyl. In some embodiments, R² is keto, and R⁴ is hydrogen. In some embodiments, R² is keto, and R⁴ is hydroxyl. In some embodiments, R² is keto, and R⁴ is mercaptan. In some embodiments, R² is keto, and R⁴ is C₁-C₃ alkyl. In some embodiments, R² is keto, and R⁴ is —CH₃.

In some embodiments, R¹ is C₁-C₃ alkyl. In some embodiments, R¹ is methyl, ethyl, n-propyl, or isopropyl. In some embodiments, R¹ is —CH₃. In some embodiments, R¹ is —CH₂CH₃. In some embodiments, R¹ is —CH₂CH₂CH₃. In some embodiments, R¹ is —CH(CH₃)₂.

In some embodiments, R¹¹ is selected from the group consisting of hydrogen or C₁-C₃ alkyl. In some embodiments, R¹¹ is selected from the group consisting of C₁-C₃ alkyl. In some embodiments, R¹¹ is hydrogen. In some embodiments, R¹¹ is methyl, ethyl, n-propyl, or isopropyl. In some embodiments, R¹¹ is hydrogen or —CH₃. In some embodiments, R¹¹ is —CH₃. In some embodiments, R¹¹ is —CH₂CH₃. In some embodiments, R¹¹ is —CH₂CH₂CH₃. In some embodiments, R¹¹ is —CH(CH₃)₂.

In some embodiments, R¹ is C₁-C₃ alkyl and R¹¹ is hydrogen. In some embodiments, R¹ is —CH₃ and R¹¹ is hydrogen.

In some embodiments, R¹ and R¹¹ are independently selected from the group consisting of C₁-C₃ alkyl. In some embodiments, one or both of R¹ and R¹¹ are —CH₃. In some embodiments, both of R¹ and R¹¹ are —CH₃.

In some embodiments, the compound of formula A is a pharmaceutically acceptable salt thereof. In some embodiments, the compound of formula (I) is a pharmaceutically acceptable salt thereof.

In some embodiments, the compound of formula (I) is a compound of formula (II):

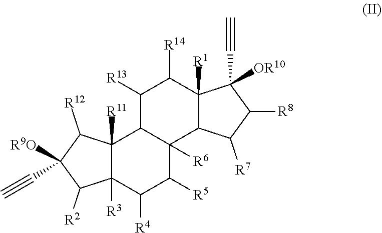

or a pharmaceutically acceptable salt thereof,
wherein R¹, R⁵, R⁶, R⁷, R⁸, R⁹, R¹⁰, R¹¹, R¹², R¹³, and R¹⁴ are as defined herein for any embodiment or variation of a compound of formula (I); R² and R⁴ are independently selected from the group consisting of hydrogen, hydroxyl, mercaptan, keto, and C₁-C₃ alkyl; and R³ is selected from the group consisting of hydrogen, hydroxyl, mercaptan, and C₁-C₃ alkyl.

In some embodiments, the compound of formula (II) is a compound of formula (II-A):

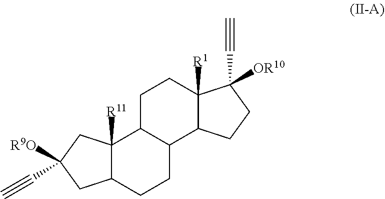

or a pharmaceutically acceptable salt thereof,
wherein R¹, R⁹, R¹⁰, and R¹¹ are as defined herein for any embodiment or variation of a compound of formula (II). In some embodiments, R¹ is —CH₃. In some embodiments, R¹¹ is —CH₃.

In some embodiments, the compound of formula (II-A) is a compound of formula (II-A-1):

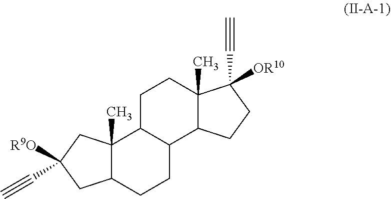

or a pharmaceutically acceptable salt thereof,
wherein R⁹ and R¹⁰ are as defined herein for any embodiment or variation of a compound of formula (II). In some embodiments, R⁹ is hydrogen. In some embodiments, R⁹ is —C(═O)CH₂CH₃. In some embodiments, R¹⁰ is hydrogen. In some embodiments, R¹⁰ is —C(═O)CH₂CH₃.

In some embodiments, the compound of formula (II-A-1) is a compound of formula (II-A-1-a):

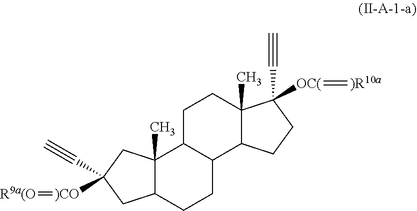

or a pharmaceutically acceptable salt thereof,
wherein R^{9a} and R^{10a} are as defined herein for any embodiment or variation of a compound of formula (II). In some embodiments, R^{9a} is —CH₂CH₃. In some embodiments, R^{10a} is —CH₂CH₃.

In some embodiments, the compound of formula (II) is a compound of formula (II-B):

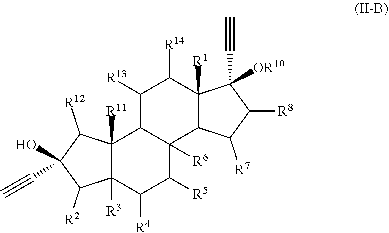

or a pharmaceutically acceptable salt thereof,
wherein R¹, R², R³, R⁴, R⁵, R⁶, R⁷, R⁸, R¹¹, R¹², R¹³, and R¹⁴ are as defined herein for any embodiment or variation of a compound of formula (II). In some embodiments, R¹ is —CH₃. In some embodiments, R¹¹ is —CH₃. In some embodiments, R², R³, R⁴, R⁵, R⁶, R⁷, R⁸, R¹², R¹³, and R¹⁴ are hydrogen.

In some embodiments, the compound of formula (II) is a compound of formula (II-C):

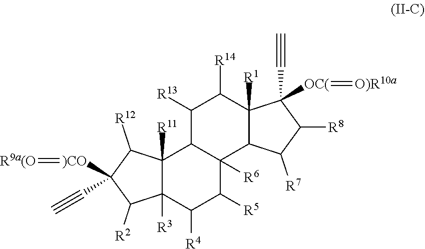

or a pharmaceutically acceptable salt thereof,
wherein R¹, R², R³, R⁴, R⁵, R⁶, R⁷, R⁸, R^{9a}, R^{10a}, R¹¹, R¹², R¹³, and R¹⁴ are as defined herein for any embodiment or variation of a compound of formula (II). In some embodiments, R¹ is —CH₃. In some embodiments, R¹¹ is —CH₃. In some embodiments, R², R³, R⁴, R⁵, R⁶, R⁷, R⁸, R¹², R¹³, and R¹⁴ are hydrogen. In some embodiments, R^{9a} is —CH₂CH₃. In some embodiments, R^{10a} is —CH₂CH₃.

In some embodiments, the compound of formula (II-C) is a compound of formula (II-C-1):

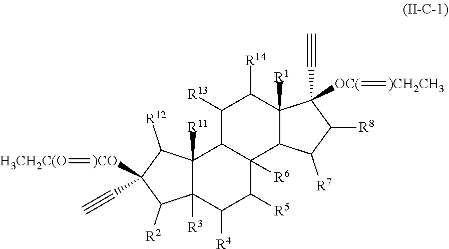

or a pharmaceutically acceptable salt thereof,
wherein R¹, R², R³, R⁴, R⁵, R⁶, R⁷, R⁸, R¹¹, R¹², R¹³, and R¹⁴ are as defined herein for any embodiment or variation of a compound of formula (II). In some embodiments, R¹ is —CH₃. In some embodiments, R¹¹ is —CH₃. In some embodiments, R², R³, R⁴, R⁵, R⁶, R⁷, R⁸, R¹², R¹³, and R¹⁴ are hydrogen.

In some embodiments, the compound of formula (I) is a compound of formula (III):

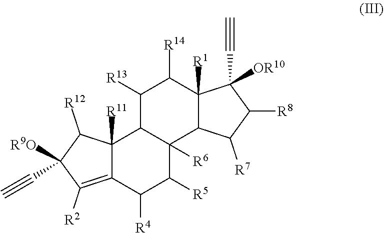

or a pharmaceutically acceptable salt thereof,
wherein R¹, R⁵, R⁶, R⁷, R⁸, R⁹, R¹⁰, R¹¹, R¹², R¹³, and R¹⁴ are as defined herein for any embodiment or variation of a compound of formula (I); R² is selected from the group consisting of hydrogen, hydroxyl, mercaptan, and C₁-C₃ alkyl; and R⁴ is selected from the group consisting of hydrogen, hydroxyl, mercaptan, keto, and C₁-C₃ alkyl.

In some embodiments, the compound of formula (III) is a compound of formula (III-A):

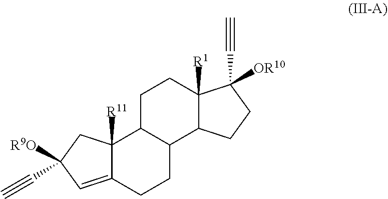

or a pharmaceutically acceptable salt thereof,
wherein R¹, R⁹, R¹⁰, and R¹¹ are as defined herein for any embodiment or variation of a compound of formula (III). In some embodiments, R¹ is —CH₃. In some embodiments, R¹¹ is —CH₃.

In some embodiments, the compound of formula (III-A) is a compound of formula (III-A-1):

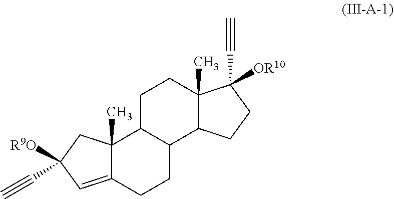

or a pharmaceutically acceptable salt thereof,
wherein R⁹ and R¹⁰ are as defined herein for any embodiment or variation of a compound of formula (III). In some embodiments, R⁹ is hydrogen. In some embodiments, R⁹ is —C(═O)CH₂CH₃. In some embodiments, R¹⁰ is hydrogen. In some embodiments, R¹⁰ is —C(═O)CH₂CH₃.

In some embodiments, the compound of formula (III-A-1) is a compound of formula (III-A-1-a):

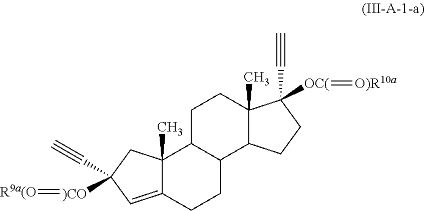

or a pharmaceutically acceptable salt thereof,
wherein R^{9a} and R^{10a} are as defined herein for any embodiment or variation of a compound of formula (III). In some embodiments, R^{9a} is —CH₂CH₃. In some embodiments, R^{10a} is —CH₂CH₃.

In some embodiments, the compound of formula (III) is a compound of formula (III-B):

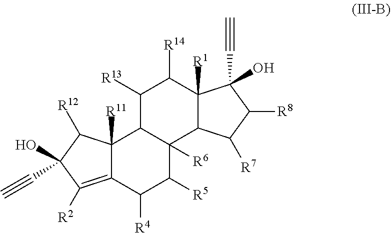

or a pharmaceutically acceptable salt thereof,
wherein R¹, R², R⁴, R⁵, R⁶, R⁷, R⁸, R¹¹, R¹², R¹³, and R¹⁴ are as defined herein for any embodiment or variation of a compound of formula (III). In some embodiments, R¹ is —CH₃. In some embodiments, R¹¹ is —CH₃. In some embodiments, R², R⁴, R⁵, R⁶, R⁷, R⁸, R¹², R¹³, and R¹⁴ are hydrogen.

In some embodiments, the compound of formula (III) is a compound of formula (III-C):

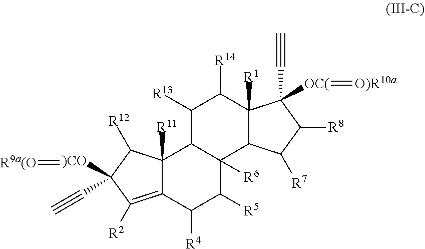

or a pharmaceutically acceptable salt thereof,
wherein R¹, R², R⁴, R⁵, R⁶, R⁷, R⁸, R^{9a}, R^{10a}, R¹¹, R¹², R¹³, and R¹⁴ are as defined herein for any embodiment or variation of a compound of formula (III). In some embodiments, R¹ is —CH₃. In some embodiments, R¹¹ is —CH₃. In some embodiments, R², R⁴, R⁵, R⁶, R⁷, R⁸, R¹², R¹³, and R¹⁴ are hydrogen. In some embodiments, R^{9a} is —CH₂CH₃. In some embodiments, R^{10a} is —CH₂CH₃.

In some embodiments, the compound of formula (III-C) is a compound of formula (III-C-1):

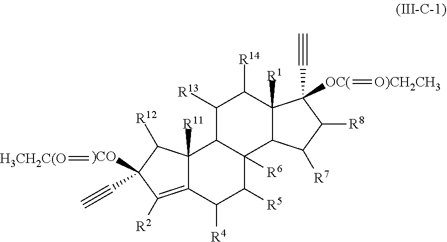

or a pharmaceutically acceptable salt thereof,
wherein R¹, R², R⁴, R⁵, R⁶, R⁷, R⁸, R¹¹, R¹², R¹³, and R¹⁴ are as defined herein for any embodiment or variation of a compound of formula (III). In some embodiments, R¹ is —CH₃. In some embodiments, R¹¹ is —CH₃. In some embodiments, R², R⁴, R⁵, R⁶, R⁷, R⁸, R¹², R¹³, and R¹⁴ are hydrogen.

In some embodiments, the compound of formula (I) is a compound of formula (IV):

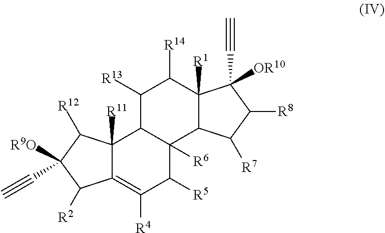

or a pharmaceutically acceptable salt thereof,
wherein R¹, R⁵, R⁶, R⁷, R⁸, R⁹, R¹⁰, R¹¹, R¹², R¹³, and R¹⁴ are as defined herein for any embodiment or variation of a compound of formula (I); R² is selected from the group consisting of hydrogen, hydroxyl, mercaptan, keto, and C₁-C₃ alkyl; and R⁴ is selected from the group consisting of hydrogen, hydroxyl, mercaptan, and C₁-C₃ alkyl.

In some embodiments, the compound of formula (IV) is a compound of formula (IV-A):

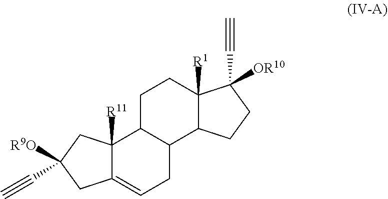

or a pharmaceutically acceptable salt thereof,
wherein R¹, R⁹, R¹⁰, and R¹¹ are as defined herein for any embodiment or variation of a compound of formula (IV). In some embodiments, R¹ is —CH₃. In some embodiments, R¹¹ is —CH₃.

In some embodiments, the compound of formula (IV-A) is a compound of formula (IV-A-1):

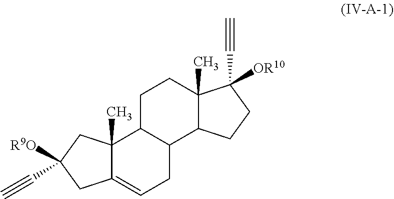

or a pharmaceutically acceptable salt thereof,
wherein R⁹ and R¹⁰ are as defined herein for any embodiment or variation of a compound of formula (IV). In some embodiments, R⁹ is hydrogen. In some embodiments, R⁹ is —C(═O)CH₂CH₃. In some embodiments, R¹⁰ is hydrogen. In some embodiments, R¹⁰ is —C(═O)CH₂CH₃.

In some embodiments, the compound of formula (IV-A-1) is a compound of formula (IV-A-1-a):

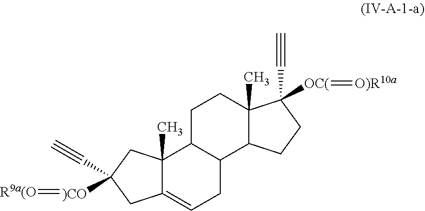

or a pharmaceutically acceptable salt thereof,
wherein R^{9a} and R^{10a} are as defined herein for any embodiment or variation of a compound of formula (IV). In some embodiments, R^{9a} is —CH₂CH₃. In some embodiments, R^{10a} is —CH₂CH₃.

In some embodiments, the compound of formula (IV) is a compound of formula (IV-B):

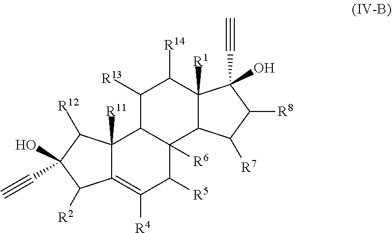

or a pharmaceutically acceptable salt thereof,
wherein R¹, R², R⁴, R⁵, R⁶, R⁷, R⁸, R¹¹, R¹², R¹³, and R¹⁴ are as defined herein for any embodiment or variation of a compound of formula (IV). In some embodiments, R¹ is —CH₃. In some embodiments, R¹¹ is —CH₃. In some embodiments, R², R⁴, R⁵, R⁶, R⁷, R⁸, R¹², R¹³, and R¹⁴ are hydrogen.

In some embodiments, the compound of formula (IV) is a compound of formula (IV-C):

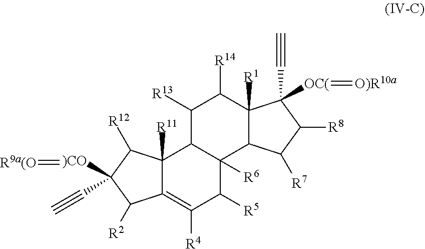

or a pharmaceutically acceptable salt thereof,
wherein R¹, R², R⁴, R⁵, R⁶, R⁷, R⁸, R^{9a}, R^{10a}, R¹¹, R¹², R¹³, and R¹⁴ are as defined herein for any embodiment or variation of a compound of formula (IV). In some embodiments, R¹ is —CH₃. In some embodiments, R¹¹ is —CH₃. In some embodiments, R², R⁴, R⁵, R⁶, R⁷, R⁸, R¹², R¹³, and R¹⁴ are hydrogen. In some embodiments, R^{9a} is —CH₂CH₃. In some embodiments, R^{10a} is —CH₂CH₃.

In some embodiments, the compound of formula (IV-C) is a compound of formula (IV-C-1):

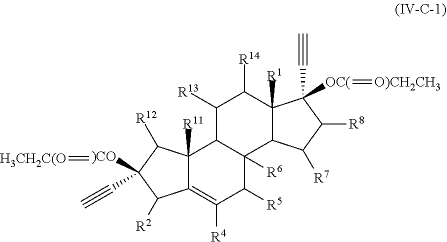

or a pharmaceutically acceptable salt thereof,
wherein R¹, R², R⁴, R⁵, R⁶, R⁷, R⁸, R¹¹, R¹², R¹³, and R¹⁴ are as defined herein for any embodiment or variation of a compound of formula (IV). In some embodiments, R¹ is —CH₃. In some embodiments, R¹¹ is —CH₃. In some embodiments, R², R⁴, R⁵, R⁶, R⁷, R⁸, R¹², R¹³, and R¹⁴ are hydrogen.

In some embodiments, R⁵, R⁷, R⁸, R¹², R¹³, and R¹⁴ are independently selected from the group consisting of hydrogen, hydroxyl, mercaptan, keto, and C₁-C₃ alkyl, and R⁶ is selected from the group consisting of hydrogen, hydroxyl, mercaptan, and C₁-C₃ alkyl. In some embodiments, R⁵, R⁶, R⁷, R⁸, R¹², R¹³, and R¹⁴ are independently selected from the group consisting of hydrogen, hydroxyl, mercaptan, and C₁-C₃ alkyl (i.e., methyl, ethyl, n-propyl, or isopropyl). In some embodiments, R⁵, R⁶, R⁷, R⁸, R¹², R¹³, and R¹⁴ are independently selected from the group consisting of hydrogen, hydroxyl, —CH₃, and —CH₂CH₃. In some embodiments, R⁵, R⁶, R⁷, R⁸, R¹², R¹³, and R¹⁴ are independently selected from the group consisting of hydrogen, hydroxyl, and —CH₃. In some embodiments, all of R⁵. R⁶, R⁷, R⁸, R¹², R¹³, and R¹⁴ are hydrogen. In some embodiments, six of R⁵, R⁶, R⁷, R⁸, R¹². R¹³, and R¹⁴ are hydrogen. In some embodiments, five of R⁵, R⁶, R⁷, R⁸, R¹², R¹³, and R¹⁴ are hydrogen. In some embodiments, four of R⁵, R⁶, R⁷, R⁸, R¹², R¹³, and R¹⁴ are hydrogen. In some embodiments, three of R⁵, R⁶, R⁷, R⁸, R¹², R¹³, and R¹⁴ are hydrogen. In some embodiments, two of R⁵, R⁶, R⁷, R⁸, R¹², R¹³, and R¹⁴ are hydrogen. In some embodiments, one of R⁵, R⁶, R⁷, R⁸, R¹², R¹³, and R¹⁴ is hydrogen. In some embodiments, three of R⁵, R⁶. R⁷, R⁸, R¹², R¹³, and R¹⁴ are hydroxyl. In some embodiments, two of R⁵, R⁶, R⁷, R⁸, R¹², R¹³, and R¹⁴ are hydroxyl. In some embodiments, one of R⁵, R⁶, R⁷, R⁸, R¹², R¹³, and R¹⁴ is hydroxyl. In some embodiments, R⁵ is hydroxyl. In some embodiments, R⁶ is hydroxyl. In some embodiments, R⁷ is hydroxyl. In some embodiments, R⁸ is hydroxyl. In some embodiments, R¹² is hydroxyl. In some embodiments, R¹³ is hydroxyl. In some embodiments, R¹⁴ is hydroxyl.

In some embodiments, three of R⁵, R⁶, R⁷, R⁸, R¹², R¹³, and R¹⁴ are mercaptan. In some embodiments, two of R⁵, R⁶, R⁷, R⁸, R¹², R¹³, and R¹⁴ are mercaptan. In some embodiments, one of R⁵, R⁶, R⁷, R⁸, R¹², R¹³, and R¹⁴ is mercaptan. In some embodiments, R⁵ is mercaptan. In some embodiments, R⁶ is mercaptan. In some embodiments, R⁷ is mercaptan. In some embodiments, R⁸ is mercaptan. In some embodiments, R¹² is mercaptan. In some embodiments, R¹³ is mercaptan. In some embodiments, R¹⁴ is mercaptan.

In some embodiments, three of R⁵, R⁷, R⁸, R¹², R¹³, and R¹⁴ are keto. In some embodiments, two of R⁵, R⁷, R⁸, R¹², R¹³, and R¹⁴ are keto. In some embodiments, one of R⁵. R⁷, R⁸, R¹², R¹³, and R¹⁴ is keto. In some embodiments, R⁵ is keto. In some embodiments, R⁷ is keto. In some embodiments, R⁸ is keto. In some embodiments, R¹² is keto. In some embodiments, R¹³ is keto. In some embodiments, R¹⁴ is keto.

In some embodiments, all of R⁵, R⁶, R⁷, R⁸, R¹², R¹³, and R¹⁴ are independently C₁-C₃ alkyl. In some embodiments, six of R⁵, R⁶, R⁷, R⁸, R¹², R¹³, and R¹⁴ are independently C₁-C₃ alkyl. In some embodiments, five of R⁵, R⁶, R⁷, R⁸, R¹², R¹³, and R¹⁴ are independently C₁-C₃ alkyl. In some embodiments, four of R⁵, R⁶, R⁷, R⁸, R¹², R¹³, and R¹⁴ are independently C₁-C₃ alkyl. In some embodiments, three of R⁵, R⁶, R⁷, R⁸, R¹², R¹³, and R¹⁴ are independently C₁-C₃ alkyl. In some embodiments, two of R⁵, R⁶, R⁷, R⁸, R¹², R¹³, and R¹⁴ are independently C₁-C₃ alkyl. In some embodiments, one of R⁵, R⁶, R⁷, R⁸, R¹², R¹³, and R¹⁴ is C₁-C₃ alkyl. In some embodiments, R⁵ is —CH₃ or —CH₂CH₃. In some embodiments, R⁶ is —CH₃ or —CH₂CH₃. In some embodiments, R⁷ is —CH₃ or —CH₂CH₃. In some embodiments, R⁸ is —CH₃ or —CH₂CH₃. In some embodiments, R¹² is —CH₃ or —CH₂CH₃. In some embodiments, R¹³ is —CH₃ or —CH₂CH₃. In some embodiments, R¹⁴ is —CH₃ or —CH₂CH₃.

In some embodiments, three of R⁵, R⁶, R⁷, R⁸, R¹², R¹³, and R¹⁴ are hydroxyl, and the others are each hydrogen. In some embodiments, two of R⁵, R⁶, R⁷, R⁸, R¹², R¹³, and R¹⁴ are hydroxyl, and the others are each hydrogen. In some embodiments, one of R⁵, R⁶, R⁷, R⁸, R¹². R¹³, and R¹⁴ is hydroxyl, and the others are each hydrogen. In some embodiments, three of R⁵, R⁶, R⁷, R⁸, R¹², R¹³, and R¹⁴ are mercaptan, and the others are each hydrogen. In some embodiments, two of R⁵, R⁶, R⁷, R⁸, R¹², R¹³, and R¹⁴ are mercaptan, and the others are each hydrogen. In some embodiments, one of R⁵, R⁶, R⁷, R⁸, R¹², R¹³, and R¹⁴ is mercaptan, and the others are each hydrogen. In some embodiments, three of R⁵, R⁶, R⁷, R⁸, R¹², R¹³, and R¹⁴ are independently C₁-C₃ alkyl, and the others are each hydrogen. In some embodiments, two of R⁵, R⁶, R⁷, R⁸, R¹², R¹³, and R¹⁴ are independently C₁-C₃ alkyl, and the others are each hydrogen. In some embodiments, one of R⁵, R⁶, R⁷, R⁸, R¹², R¹³, and R¹⁴ is C₁-C₃ alkyl, and the others are each hydrogen. In some embodiments, one of R⁵, R⁶, R⁷, R⁸, R¹², R¹³, and R¹⁴ is hydroxyl, one of R⁵, R⁶, R⁷, R⁸, R¹², R¹³, and R¹⁴ is C₁-C₃ alkyl, and the others are each hydrogen. In some embodiments, one of R⁵, R⁶, R⁷, R⁸, R¹², R¹³, and R¹⁴ is mercaptan, one of R⁵, R⁶, R⁷, R⁸, R¹², R¹³, and R¹⁴ is C₁-C₃ alkyl, and the others are each hydrogen. It is understood that when two or more R⁵, R⁶, R⁷, R⁸, R¹², R¹³, and R¹⁴ are C₁-C₃ alkyl, each C₁-C₃ alkyl is independently methyl, ethyl, n-propyl, or isopropyl.

In some embodiments, any one or more of R², R³, R⁴, R⁵, R⁶, R⁷, R⁸, R¹¹, R¹², R¹³, and R¹⁴ are hydrogen. In some embodiments, all of R², R³, R⁴, R⁵, R⁶, R⁷, R⁸, R¹¹, R¹², R¹³, and R¹⁴ are hydrogen. In some embodiments, any one or more of R², R³, R⁴, R⁵, R⁶, R⁷, R⁸, R¹², R¹³, and R¹⁴ are hydrogen. In some embodiments, all of R², R³, R⁴, R⁵, R⁶, R⁷, R⁸, R¹². R¹³, and R¹⁴ are hydrogen.

In some embodiments, R², R³, R⁴, R⁵, R⁶, R⁷, R⁸, R¹², R¹³, and R¹⁴ are independently in the α- or β-configuration. In some embodiments, all of R², R³, R⁴, R⁵, R⁶, R⁷, R⁸, R¹², R¹³ and R¹⁴ are in the α-configuration. In some embodiments, nine of R², R³, R⁴, R⁵, R⁶, R⁷, R⁸, R¹², R¹³, and R¹⁴ are in the α-configuration. In some embodiments, eight of R², R³, R⁴, R⁵, R⁶. R⁷, R⁸, R¹², R¹³, and R¹⁴ are in the α-configuration. In some embodiments, seven of R², R³. R⁴, R⁵, R⁶, R⁷, R⁸, R¹², R¹³, and R¹⁴ are in the α-configuration. In some embodiments, six of R², R³, R⁴, R⁵, R⁶, R⁷, R⁸, R¹², R¹³, and R¹⁴ are in the α-configuration. In some embodiments, five of R², R³, R⁴, R⁵, R⁶, R⁷, R⁸, R¹², R¹³, and R¹⁴ are in the α-configuration. In some embodiments, four of R², R³, R⁴, R⁵, R⁶, R⁷, R⁸, R¹², R¹³, and R¹⁴ are in the α-configuration. In some embodiments, three of R², R³, R⁴, R⁵, R⁶, R⁷, R⁸, R¹², R¹³, and R¹⁴ are in the α-configuration. In some embodiments, two of R², R³, R⁴, R⁵, R⁶, R⁷, R⁸, R¹², R¹³, and R¹⁴ are in the α-configuration. In some embodiments, one of R², R³, R⁴, R⁵, R⁶, R⁷, R⁸, R¹², R¹³, and R¹⁴ is in the α-configuration. In some embodiments, all of R², R³, R⁴, R⁵, R⁶, R⁷, R⁸, R¹², R¹³ and R¹⁴ are in the β-configuration. In some embodiments, nine of R², R³, R⁴, R⁵, R⁶, R⁷, R⁸, R¹², R¹³, and R¹⁴ are in the β-configuration. In some embodiments, eight of R², R³, R⁴, R⁵, R⁶, R⁷, R⁸, R¹², R¹³, and R¹⁴ are in the β-configuration. In some embodiments, seven of R², R³, R⁴, R⁵, R⁶, R⁷, R⁸, R¹², R¹³, and R¹⁴ are in the β-configuration. In some embodiments, six of R², R³, R⁴, R⁵, R⁶, R⁷, R⁸, R¹², R¹³, and R¹⁴ are in the β-configuration. In some embodiments, five of R², R³, R⁴, R⁵, R⁶, R⁷, R⁸, R¹², R¹³, and R¹⁴ are in the β-configuration. In some embodiments, four of R², R³, R⁴, R⁵, R⁶, R⁷, R⁸, R¹², R¹³, and R¹⁴ are in the β-configuration. In some embodiments, three of R², R³, R⁴, R⁵, R⁶, R⁷, R⁸, R¹², R¹³, and R¹⁴ are in the β-configuration. In some embodiments, two of R², R³, R⁴, R⁵, R⁶, R⁷, R⁸, R¹², R¹³, and R¹⁴ are in the β-configuration. In some embodiments, one of R², R³, R⁴, R⁵, R⁶, R⁷, R⁸, R¹², R¹³, and R¹⁴ is in the β-configuration. In some embodiments, both R³ and R⁶ are in the α-configuration. In some embodiments, R³ is in the β-configuration, and R⁶ is in the α-configuration. In some embodiments, substituents above the plane of the compound are described to be in the β-configuration and can be shown as a wedge bond. In some embodiments, substituents below the plane of the compound are described to be in the α-configuration and can be shown as a dash bond.

In some embodiments, wherein [symbol] between the carbon bearing R² and the carbon bearing R³ represents a single bond, and wherein R² is selected from the group consisting of hydroxyl, mercaptan, and C₁-C₃ alkyl, R² is in the α- or β-configuration. In some embodiments, R² is in the α-configuration. In some embodiments, R² is in the β-configuration.

In some embodiments, wherein [symbol] between the carbon bearing R² and the carbon bearing R³ represents a single bond, wherein [symbol] between the carbon bearing R³ and the carbon bearing R⁴ represents a single bond, and wherein R³ is selected from the group consisting of hydrogen, hydroxyl, mercaptan, and C₁-C₃ alkyl, R³ is in the α- or β-configuration. In some embodiments, R³ is in the α-configuration. In some embodiments, R³ is in the β-configuration.

In some embodiments, wherein [symbol] between the carbon bearing R³ and the carbon bearing R⁴ represents a single bond, and wherein R⁴ is selected from the group consisting of hydroxyl, mercaptan, and C₁-C₃ alkyl, R⁴ is in the α- or β-configuration. In some embodiments, R⁴ is in the α-configuration. In some embodiments, R⁴ is in the β-configuration.

In some embodiments, wherein R⁵ is selected from the group consisting of hydroxyl, mercaptan, and C₁-C₃ alkyl, R⁵ is in the α- or β-configuration. In some embodiments, R⁵ is in the α-configuration. In some embodiments, R⁵ is in the β-configuration.

In some embodiments, wherein R⁶ is selected from the group consisting of hydrogen, hydroxyl, mercaptan, and C₁-C₃ alkyl, R⁶ is in the α- or β-configuration. In some embodiments, R⁶ is in the α-configuration. In some embodiments, R⁶ is in the β-configuration.

In some embodiments, wherein R⁷ is selected from the group consisting of hydroxyl, mercaptan, and C₁-C₃ alkyl, R⁷ is in the α- or β-configuration. In some embodiments, R⁷ is in the α-configuration. In some embodiments, R⁷ is in the β-configuration.

In some embodiments, wherein R⁸ is selected from the group consisting of hydroxyl, mercaptan, and C₁-C₃ alkyl, R⁸ is in the α- or β-configuration. In some embodiments, R⁸ is in the α-configuration. In some embodiments, R⁸ is in the β-configuration.

In some embodiments, wherein R¹² is selected from the group consisting of hydroxyl, mercaptan, and C₁-C₃ alkyl, R¹² is in the α- or β-configuration. In some embodiments, R¹² is in the α-configuration. In some embodiments, R¹² is in the β-configuration.

In some embodiments, wherein R¹³ is selected from the group consisting of hydroxyl, mercaptan, and C₁-C₃ alkyl, R¹³ is in the α- or β-configuration. In some embodiments, R¹³ is in the α-configuration. In some embodiments, R¹³ is in the β-configuration.

In some embodiments, wherein R¹⁴ is selected from the group consisting of hydroxyl, mercaptan, and C₁-C₃ alkyl, R¹⁴ is in the α- or β-configuration. In some embodiments, R¹⁴ is in the α-configuration. In some embodiments, R¹⁴ is in the β-configuration.

In some embodiments, R⁹ is selected from the group consisting of hydrogen and —C(═O)R^{9a}. In some embodiments, R⁹ is —C(═O)R^{9a}, wherein R^{9a} is selected from the group consisting of C₁-C₆ alkyl and C₂-C₆ alkenyl, and wherein the C₁-C₆ alkyl and C₂-C₆ alkenyl are optionally substituted with —C(═O)OH.

In some embodiments, R⁹ is hydrogen.

In some embodiments, R⁹ is —C(═O)R^{9a}, wherein R^{9a} is C₁-C₆ alkyl optionally substituted with —C(═O)OH.

In some embodiments, R⁹ is —C(═O)R^{9a}, wherein R^{9a} is unsubstituted C₁-C₆ alkyl (i.e., C₁ alkyl, C₂ alkyl, C₃ alkyl, C₄ alkyl, C₅ alkyl, or C₆ alkyl). In some embodiments, R⁹ is —C(═O)R^{9a}, wherein R^{9a} is unsubstituted C₁-C₃ alkyl (i.e., methyl, ethyl, n-propyl, or isopropyl). In some embodiments, R⁹ is —C(═O)R^{9a}, wherein R^{9a} is —CH₂CH₃. In some embodiments, R⁹ is —C(═O)R^{9a}, wherein R^{9a} is —CH₃.

In some embodiments, R⁹ is —C(═O)R^{9a}, wherein R^{9a} is C₁-C₆ alkyl (i.e., C₁ alkyl, C₂ alkyl, C₃ alkyl, C₄ alkyl, C₅ alkyl, or C₆ alkyl) substituted with —C(═O)OH. In some embodiments, R⁹ is —C(═O)R^{9a}, wherein R^{9a} is C₁-C₃ alkyl (i.e., methyl, ethyl, n-propyl, or isopropyl) substituted with —C(═O)OH. In some embodiments, R⁹ is —C(═O)R^{9a}, wherein R^{9a} is —CH₂C(═O)OH, —CH₂CH₂C(═O)OH, —CH(C(═O)OH)CH₃, —CH₂CH₂CH₂C(═O)OH, —CH₂CH(C(═O)OH)CH₃, —CH(C(═O)OH)CH₂CH₃, —CH(CH₃)CH₂C(═O)OH, or —C(CH₃)₂C(═O)OH. In some embodiments, R⁹ is —C(═O)R^{9a}, wherein R^{9a} is —CH₂CH₂C(═O)OH.

In some embodiments, R⁹ is —C(═O)R^{9a}, wherein R^{9a} is C₂-C₆ alkenyl optionally substituted with —C(═O)OH.

In some embodiments, R⁹ is —C(═O)R^{9a}, wherein R^{9a} is unsubstituted C₂-C₆ alkenyl (i.e., C₂ alkenyl, C₃ alkenyl, C₄ alkenyl, C₅ alkenyl, or C₆ alkenyl). In some embodiments, R⁹ is —C(═O)R^{9a}, wherein R^{9a} is unsubstituted C₄-C₆ alkenyl containing one or two carbon-carbon double bonds (e.g., —CH═CHCH₂CH₃, —CH═CHCH₂CH₂CH₃, —CH═CHCH₂CH═CH₂CH₃, or —CH═CHCH₂CH₂). It is understood that internal alkenyls can have E or Z configuration. For example, —CH═CHCH₂CH₃ can be

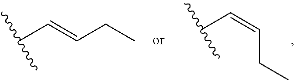

wherein the wavy line indicates point of attachment to the remainder of the molecule. In some embodiments, R^{9a} is unsubstituted C₄-C₅ alkenyl containing one carbon-carbon double bond (e.g., —CH═CHCH₂CH₃, —CH₂CH═CHCH₃, —CH₂CH₂CH═CH₂, —CH═CHCH₂CH₂CH₃, —CH₂CH═CHCH₂CH₃, —CH₂CH₂CH═CHCH₃, —CH₂CH₂CH₂CH═CH₂, or —CH═CHCH(CH₃)₂. In some embodiments, R^{9a} is unsubstituted C₆ alkenyl containing one or two carbon-carbon double bonds (e.g., —CH═CHCH₂CH₂CH₂CH₃, —CH₂CH═CHCH₂CH₂CH₃, —CH₂CH₂CH═CHCH₂CH₃, —CH₂CH₂CH₂CH═CHCH₃, —CH₂CH₂CH₂CH₂CH═CH₂, —CH═CHCH₂CH(CH₃)₂, —CH═CHCH═CHCH₂CH₃, —CH═CHCH₂CH═CHCH₃, or —CH═CHCH₂CH₂CH═CH₂).

In some embodiments, R⁹ is —C(═O)R^{9a}, wherein R^{9a} is C₂-C₆ alkenyl (i.e., C₂ alkenyl, C₃ alkenyl, C₄ alkenyl, C₅ alkenyl, or C₆ alkenyl) substituted with —C(═O)OH. In some embodiments, R⁹ is —C(═O)R^{9a}, wherein R^{9a} is C₂-C₄ alkenyl (e.g., ethenyl or propenyl) substituted with —C(═O)OH. In some embodiments, R⁹ is —C(═O)R^{9a}, wherein R^{9a} is —CH═CHC(═O)OH, —CH═CHCH₂C(═O)OH, —CH₂CH═CHC(═O)OH, or —CH═C(CH₃)C(═O)OH. In some embodiments, R⁹ is —C(═O)R^{9a}, wherein R^{9a} is —CH═CHC(═O)OH.

In some embodiments, R¹⁰ is selected from the group consisting of hydrogen and —C(═O)R^{10a}. In some embodiments, R¹⁰ is —C(═O)R^{10a}, wherein R^{10a} is selected from the group consisting of C₁-C₆ alkyl and C₂-C₆ alkenyl, and wherein the C₁-C₆ alkyl and C₂-C₆ alkenyl are optionally substituted with —C(═O)OH.¹⁰

In some embodiments, R¹⁰ is hydrogen.

In some embodiments, R¹⁰ is —C(═O)R^{10a}, wherein R^{10a} is C₁-C₆ alkyl optionally substituted with —C(═O)OH.

In some embodiments, R¹⁰ is —C(═O)R^{10a}, wherein R^{10a} is unsubstituted C₁-C₆ alkyl (i.e., C₁ alkyl, C₂ alkyl, C₃ alkyl, C₄ alkyl, C₅ alkyl, or C₆ alkyl). In some embodiments, R¹⁰ is —C(═O)R^{10a}, wherein R^{10a} is unsubstituted C₁-C₃ alkyl (i.e., methyl, ethyl, n-propyl, or isopropyl). In some embodiments, R¹⁰ is —C(═O)R^{10a}, wherein R^{10a} is —CH₂CH₃. In some embodiments, R¹⁰ is —C(═O)R^{10a}, wherein R^{10a} is —CH₃.

In some embodiments, R¹⁰ is —C(═O)R^{10a}, wherein R^{10a} is C₁-C₆ alkyl (i.e., C₁ alkyl, C₂ alkyl, C₃ alkyl, C₄ alkyl, C₅ alkyl, or C₆ alkyl) substituted with —C(═O)OH. In some embodiments, R¹⁰ is —C(═O)R^{10a}, wherein R^{10a} is C₁-C₃ alkyl (i.e., methyl, ethyl, n-propyl, or isopropyl) substituted with —C(═O)OH. In some embodiments, R¹⁰ is —C(═O)R^{10a}, wherein R^{10a} is —CH₂C(═O)OH, —CH₂CH₂C(═O)OH, —CH(C(═O)OH)CH₃, —CH₂CH₂CH₂C(═O)OH, —CH₂CH(C(═O)OH)CH₃, —CH(C(═O)OH)CH₂CH₃, —CH(CH₃)CH₂C(═O)OH, or —C(CH₃)₂C(═O)OH. In some embodiments, R¹⁰ is —C(═O)R^{10a}, wherein R^{10a} is —CH₂CH₂C(═O)OH.

In some embodiments, R¹⁰ is —C(═O)R^{10a}, wherein R^{10a} is C₂-C₆ alkenyl optionally substituted with —C(═O)OH.

In some embodiments, R¹⁰ is —C(═O)R^{10a}, wherein R^{10a} is unsubstituted C₂-C₆ alkenyl (i.e., C₂ alkenyl, C₃ alkenyl, C₄ alkenyl, C₅ alkenyl, or C₆ alkenyl). In some embodiments, R¹⁰ is —C(═O)R^{10a}, wherein R^{10a} is unsubstituted C₄-C₆ alkenyl containing one or two carbon-carbon double bonds (e.g., —CH═CHCH₂CH₃, —CH═CHCH₂CH₂CH₃, —CH═CHCH₂CH₂CH₂CH₃, or —CH═CHCH₂CH₂). In some embodiments, R^{10a} is unsubstituted C₄-C₅ alkenyl containing one carbon-carbon double bond (e.g., —CH═CHCH₂CH₃, —CH₂CH═CHCH₃, —CH₂CH₂CH═CH₂, —CH═CHCH₂CH₂CH₃, —CH₂CH═CHCH₂CH₃, —CH₂CH₂CH═CHCH₃, —CH₂CH₂CH₂CH═CH₂, or —CH═CHCH(CH₃)₂. In some embodiments, R^{10a} is unsubstituted C₆ alkenyl containing one or two carbon-carbon double bonds (e.g., —CH═CHCH₂CH₂CH₂CH₃, —CH₂CH═CHCH₂CH₂CH₃, —CH₂CH₂CH═CHCH₂CH₃, —CH₂CH₂CH₂CH═CHCH₃, —CH₂CH₂CH₂CH₂CH═CH₂, —CH═CHCH₂CH(CH₃)₂, —CH═CHCH═CHCH₂CH₃, —CH═CHCH₂CH═CHCH₃, or —CH═CHCH₂CH₂CH═CH₂).

In some embodiments, R¹⁰ is —C(═O)R^{10a}, wherein R^{10a} is C₂-C₆ alkenyl (i.e., C₂ alkenyl, C₃ alkenyl, C₄ alkenyl, C₅ alkenyl, or C₆ alkenyl) substituted with —C(═O)OH. In some embodiments, R¹⁰ is —C(═O)R^{10a}, wherein R^{10a} is C₂-C₄ alkonyl (e.g., ethenyl or propenyl) substituted with —C(═O)OH. In some embodiments, R¹⁰ is —C(═O)R^{10a}, wherein R^{10a} is —CH═CHC(═O)OH, —CH═CHCH₂C(═O)OH, —CH₂CH═CHC(═O)OH, or —CH═C(CH₃)C(═O)OH. In some embodiments, R¹⁰ is —C(═O)R^{10a}, wherein R^{10a} is —CH═CHC(═O)OH.

In some embodiments, R⁹ and R¹⁰ are independently hydrogen or —C(═O)CH₂CH₃. In some embodiments, R⁹ and R¹⁰ are each hydrogen. In some embodiments, R⁹ and R¹⁰ are each —C(═O)CH₂CH₃. In some embodiments, R⁹ is hydrogen and R¹⁰ is —C(═O)CH₂CH₃. In some embodiments, R⁹ is —C(═O)CH₂CH₃ and R¹⁰ is hydrogen.

### Anordrin

2β, 7α-diethyl-A-nor-5α-androstane-2α, 17β-diol (anordiol) was first reported as possessing anti-estrogenic activity. See Bank et al, Proc. Soc. Expt. Biol. Med. 111, 595 (1962) and Pincus et al, Steroids, 5, 193, (1965). Li et al esterified anordiol using propionic acid to synthesize 2α, 17α-diethynyl-A-nor-5α-androstane-2β,17β-diol dipropionate (anordrin, ANO) in 1969. Anordrin was marketed as an antifertility medicine using the brand name AF-53 in China beginning in 1976. Estrogen is known to cause hormone-induced cancer, and anordrin, as an estrogen receptor antagonist, was subsequently found to inhibit malignant cell growth and treat cancer. See Xu et al, Tumor, 9, 197 (1989); Ma et al, Acta Pharmacol. Sin, 21, 939 (2000); Li et al, U.S. Pat. No. 5,001,120 (1991).

In some embodiments, the anordrin or analog thereof is anordrin.

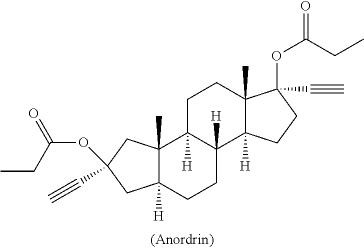

### Insomnia

Insomnia is one of the most common symptoms for which adults seek medical advice. Insomnia can be caused by various factors. Insomnia described in the present application in some embodiments is insomnia associated with a low level of estrogen. In some embodiments, the insomnia is associated with perimenopause, menopause, or postmenopause.

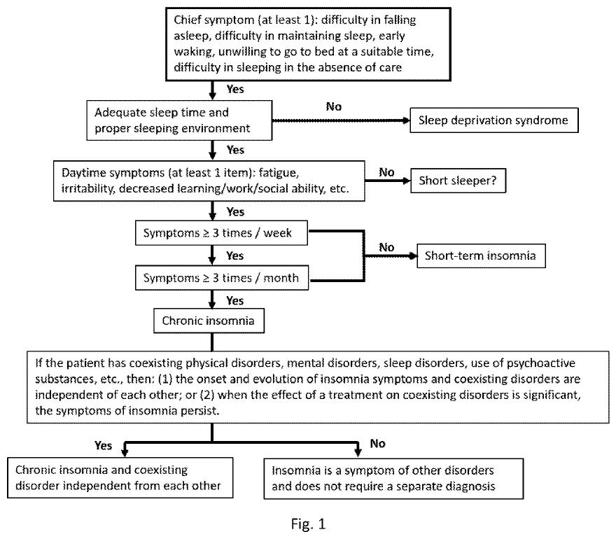

Insomnia is usually measured using sleep disorder questionnaire (SDQ) such as Pittsburgh sleep quality index (PSQI), Epworth Sleeping Scale or the Athens Insomnia Scale (AIS), or using sleep quality monitor to count sleep dysfunction rating scale (SDRS). AIS is the one of popularly used SEQ. See FIG. 1. It is measured by assessing eight factors amongst which first five factors are related to nocturnal sleep and last three factors are related to daytime dysfunction. These are rated on a 0-3 scale and the sleep is finally evaluated from the cumulative score of all factors and reported as an individual's sleep outcome. Over the period of time, AIS is considered to be an effective tool in sleep analysis, and it is validated in various countries by testing it on local patients. A cut-off score of at least 6 on the AIS is used to establish the diagnosis of insomnia. See Soldatos C R, Dikeos D G, Paparrigopoulos T J (2002). “The diagnostic validity of the Athens Insomnia Scale”. J Psychosom Res. 55 (3): 263-7.

In some embodiments, the insomnia is chronic insomnia.

In some embodiments, the insomnia is a severe form of insomnia (e.g., the AIS score is at least about 22). In some embodiments, the insomnia is a moderate form of insomnia (e.g., the AIS score is at least about 15). In some embodiments, the AIS score is decreased by at least about 10%, 20%, 30%, 40%, 50%, or 60% after treatment at a frequency and dosage for a duration of time as described herein.

In some embodiments, the insomnia is a severe form of insomnia (e.g., the PSQI score is at least about 16). In some embodiments, the insomnia is a moderate form of insomnia (e.g., the PSQI score is at least about 11). In some embodiments, the PSQI score is decreased by at least about 10%, 20%, 30%, 40%, 50%, or 60% after treatment at a frequency and dosage for a duration of time as described herein.

### Patient Population

In some embodiments, the individual is a mammal. In some embodiments, the individual is a human.

In some embodiments, the individual is a female.

In some embodiments, the individual has thick endometrium. In some embodiments, the thickness of endometrium is no more than 6 mm, 5 mm, 4.5 mm, or 4 mm.

In some embodiments, the individual has been subject to hysterectomy.

In some embodiments, the individual is at least about 35, 40, or 45 years old. In some embodiments, the individual age is about 45 to about 90 or old.

In some embodiments, the individual is at perimenopause stage (e.g., the individual still has menstrual periods but has decreased level of estrogen. In some embodiments, the individual reaches menopause. In some embodiments, the individual is at the postmenopausal stage. In some embodiments, it has been at least about 1, 2, 3, 4, 5, 6, 7, 8, 9, 10, 11, or 12 months since the last period episode.

In some embodiments, the individual has been subject to a surgical removal of ovary.

In some embodiments, the individual has not been subject to a hormone therapy. In some embodiments, the individual has been subject to a hormone therapy.

In some embodiments, the individual has a cancer or previously had a cancer. In some embodiments, the cancer is a breast cancer, ovarian cancer or endometrial cancer.

In some embodiments, the individual has a stroke or previously had a stroke, liver disease, blood clots in legs or lungs, or unexplained vaginal bleeding.

In some embodiments, the individual has a severe form of insomnia (e.g., the AIS score is at least about 22). In some embodiments, the individual has a moderate form of insomnia (e.g., the AIS score is at least about 15). In some embodiments, the individual has an AIS score of at least about 6. In some embodiments, the individual has an AIS score of at least about 7. In some embodiments, the individual has an AIS score of at least about 15. In some embodiments, the individual has an AIS score of at least about 22.

In some embodiments, the individual has a severe form of insomnia (e.g., the PSQI score is at least about 16). In some embodiments, the individual has a moderate form of insomnia (e.g., the PSQI score is at least about 11). In some embodiments, the individual has a PSQI score of at least about 11. In some embodiments, the individual has a PSQI score of at least about 16.

### Dosing and Methods of Administration

The dose of the composition comprising the compound of formula A or formula I may vary with the particular composition, the nature of the individual, the mode of administration, and the severity of insomnia. In some embodiments, the amount of the formula I is sufficient to produce an improved sleep in any one of the conditions selected from the group consisting of a) sleep induction time, b) awakening times during the night, c) if final awakening is earlier than desired and the extent of the earlier awakening, d) total sleep duration, c) overall quality of sleep, f) sense of well-being during the day, g) functioning (physical and/or mental) during the day, and h) sleepiness during the day. In some embodiments, the amount of the formula I is sufficient to produce a decrease in SDQ or SDRS score by at least about 5%. 10%, 15%. 20%, 30%, 40%, 50%, or 60%. In some embodiments, the amount of the formula I is sufficient to produce a decrease of AIS score by at least about 5%. 10%, 15%. 20%. 30%. 40%, 50%, or 60% for at least one, two, three, or four weeks. AIS score can be assessed by the individual, for example, by fulfilling the Table in FIG. 1 before and after treatment.

In some embodiments, the amount of the formula I is sufficient to induce an extracellular ATP level surge among neuronal cells (see, e.g., Example 3 and/or FIG. 5).

In some embodiments, the dose of the compound for each administration is about 1 μg to about 10 mg (such as about 10 μg to about 1 mg) for a human or a comparable dose for an individual that is not a human. In some embodiments, the dose of the compound for each administration is at least about 1 μg, 5 μg, 10 μg, 20 μg, 50 μg, 75 μg, 100 μg, 125 μg, 150 μg, 175 μg, 200 μg, 250 μg, 300 μg, 350 μg, 400 μg, 450 μg, or 500 μg for a human or a comparable dose for an individual that is not a human. In some embodiments, the dose of the compound for each administration is no more than about 10 mg, 7.5 mg, 5 mg, 2.5 mg, 2 mg. 1.5 mg, 1 mg, 0.9 mg, 0.8 mg, 0.7 mg, 0.6 mg, 0.5 mg, 0.4 mg, 0.3 mg, or 0.2 mg for a human or a comparable dose for an individual that is not a human. In some embodiments, the dose of the compound for each administration is about 20 μg to about 2 mg (such as about 20 μg to about 200 μg, or about 400 μg to about 2 mg) for a human or a comparable dose for an individual that is not a human. The comparable dose for an individual that is not a human can be calculated, for example, based upon guidance provided in Nair. A. B., & Jacob. S. (2016). A simple practice guide for dose conversion between animals and human. Journal of basic and clinical pharmacy, 7(2). 27.

In some embodiments, the dose of the compound for each administration in a human subject is about 0.01 mg to about 10 mg. In some embodiments, the dose of the compound for each administration in a human subject is about 0.01 mg to about 1 mg (e.g., about 0.05 mg to about 0.5 mg). In some embodiments, the dose of the compound for each administration in a human subject is about 0.5 mg to about 5 mg. In some embodiments, the dose of the compound for each administration in a human subject is at least about 0.01 mg, e.g., at least about 0.02 mg, e.g., at least about 0.05 mg.

In some embodiments, the dose of the compound for each administration in a human subject is about 0.05 mg. In some embodiments, the dose of the compound for each administration in a human subject is about 0.1 mg, of the compound for each administration in a human subject is about 0.15 mg.

In some embodiments, the daily dose of the compound in a human subject is about 0.01 mg to about 10 mg (e.g., about 0.05 mg to about 0.5 mg, e.g., about 0.05 mg to about 0.15 mg). In some embodiments, the weekly dose of the compound in a human subject is about 0.1 mg to about 10 mg (e.g., about 0.25 mg to about 5 mg, e.g., about 0.25 mg to about 1.2 mg).

In some embodiments, the composition is administered at a frequency of about once, twice or three times a day. In some embodiments, the composition is administered at a frequency of about or at least about once, twice or three times a week. In some embodiments, the composition is administered at a frequency of about once every two weeks.

In some embodiments, the composition is administered daily at a dosage of about 20 μg to about 200 μg (such as about 50 μg, 100 μg, or 150 μg). In some embodiments, the composition is administered weekly at a dosage of about 400 μg to about 2 mg.

In some embodiments, the composition is administered into the individual (such as at a frequency and/or dose as described herein) for at least about one, two, three, four, five, six, seven, or eight weeks. In some embodiments, the composition is administered into the individual (such as at a frequency and/or dose as described here) for at least two weeks. In some embodiments, the composition is administered into the individual (such as at a frequency and/or dose as described herein) for at least about one, two, three, four, five, six, seven, eight, nine, ten, eleven or twelve months. In some embodiments, the composition is administered into the individual (such as at a frequency and/or dose as described herein) for at least about one year, one and a half years, two years, three years, four years or five years.

The composition described herein can be administered to an individual (such as a human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the composition is administered orally.

### Pharmaceutical Composition

Further provided are pharmaceutical compositions, comprising a compound of formula A or formula I or a pharmaceutically acceptable salt or ester thereof described herein and one or more pharmaceutically acceptable carriers, excipients, stabilizing agents, diluents, and/or other agents, which are known in the art, for use in the methods described herein.

In some embodiments, there is provided a pharmaceutical composition comprising a compound of formula I (such as anordrin) or a pharmaceutically acceptable salt or ester thereof and a pharmaceutically acceptable carrier.

The pharmaceutical compositions described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the embodiments may be administered by a suitable route of delivery, such as oral, parenteral, rectal, nasal, topical, or ocular routes, or by inhalation. In some embodiments, the pharmaceutical composition is formulated for oral administration.

For oral administration, the pharmaceutical composition may be provided in a solid form, such as a tablet or capsule, or as a solution, emulsion, or suspension. In some embodiments, the pharmaceutical composition is formulated as a tablet, a capsule or a pill. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating. The oral formulations may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol. Capsules may also contain gelatin, iron oxides, stearic acid, and titanium dioxide as inactive ingredients.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

### Kits, Medicines, Compositions and Unit Dosages

The present application also provides kits, medicines, compositions, and unit dosage forms for use in any of the methods described herein.

Kits of the invention include one or more containers comprising a composition (such as a pharmaceutical composition) comprising a compound of formula A or formula I or a pharmaceutically acceptable salt or ester thereof described herein (or a unit dosage forms and/or articles of manufacture), further comprise instructions for use in accordance with any of the methods described herein. The kit may further comprise a description of selecting an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a) a composition comprising a compound of formula I (e.g., anordrin) as described herein (such as anordrin) or a pharmaceutically acceptable salt or ester thereof and b) b) instructions for administering the composition for treatment of insomnia. In some embodiments, the kit comprises a) a composition comprising anordrin and b) instructions for administering (such as orally administering) the composition for treatment of insomnia. In some embodiments, the kit further comprises a second agent. The composition and the other agents can be present in separate containers or in a single container. For example, the kit may comprise one distinct composition or two or more compositions wherein one composition comprises composition and one composition comprises another agent.

The kits of the invention are in suitable packaging. Suitable packaging include, but is not limited to, vials, bottles, jars, flexible packaging (e.g., seled Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the compound (such as anordrin) as disclosed herein to provide effective treatment of an individual for any period of time, such as any of about 1, 2, 3, 4, 5, 6, or 7 days, or about 1, 2, 3, 4, 5, or 6 week, or about 1, 2, 3, 4, 5, 6, 7, 8, 9, 10, 11, or 12 months, or 1, 2, 3, 4, 5, 6 years. Kits may also include multiple unit doses of the compound of formula I (such as anordrin) and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. In some embodiments, there is provided a medicine (or composition) for use in treating insomnia, comprising a composition comprising a compound of formula I (e.g., anordrin) as described herein (such as anordrin) or a pharmaceutically acceptable salt or ester thereof.

### EXEMPLARY EMBODIMENTS

Embodiment 1. A method of treating insomnia in an individual, comprising administering to the individual an effective amount of a composition comprising a compound of formula I:

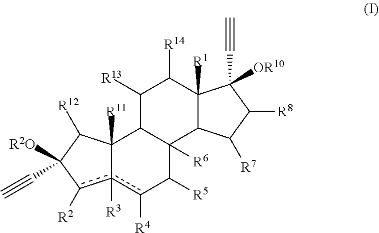

or a pharmaceutically acceptable salt or ester thereof, wherein:
a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl,
   [symbol] between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl; or
   [symbol] between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl,
b) R¹ is C₁-C₃ alkyl;
c) R⁵, R⁷, R⁸, R¹², R¹³ and R¹⁴ are independently selected from the group consisting of hydrogen, hydroxyl, mercaptan, keto, and C₁-C₃ alkyl;
d) R⁶ is selected from the group consisting of hydrogen, hydroxyl, mercaptan, and C₁-C₃ alkyl;
e) R⁹ is selected from the group consisting of hydrogen and —C(═O)R^{9a};
f) R¹⁰ is selected from the group consisting of hydrogen and —C(═O)R^{10a};
g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and
h) R¹¹ is selected from the group consisting of C₁-C₃ alkyl.
Embodiment 2. The method of embodiment 1, wherein R⁹ and R¹⁰ are independently hydrogen or —C(═O)CH₂CH₃.
Embodiment 3. The method of embodiment 1 or embodiment 2, wherein one or both of R¹ and R¹¹ are —CH₃.
Embodiment 4. The method of any one of embodiments 1-3, wherein any one or more of R², R³, R⁴, R⁵, R⁶, R⁷, R⁸, R¹², R¹³, and R¹⁴ are hydrogen.
Embodiment 5. The method of any one of embodiments 1-4, wherein the compound is anordrin.
Embodiment 6. The method of any one of embodiments 1-5, wherein the individual is a female.
Embodiment 7. The method of any one of embodiments 1-6, wherein the individual is a human.
Embodiment 8. The method of any one of embodiments 1-7, wherein the individual is at perimenopause stage, reaches menopause, is post menopause, or has been subject to a surgical removal of ovary.
Embodiment 9. The method of embodiment 8, wherein the individual is a postmenopausal woman.
Embodiment 10. The method of any one of embodiments 1-9, wherein the individual has an aberrant level of estrogen.
Embodiment 11. The method of any one of embodiments 1-10, wherein the individual has a score of at least about 7 according to Athens Insomnia Scale.
Embodiment 12. The method of any one of embodiments 1-11, wherein the individual is not subject or has not been subject to a hormone therapy.
Embodiment 13. The method of any one of embodiments 1-12, wherein the dose of the compound for each administration is at least about 20 μg for a human or a comparable dose for an individual that is not a human.
Embodiment 14. The method of embodiment 13, wherein the dose of the compound for each administration is at least about 50 μg for a human or a comparable dose for an individual that is not a human.
Embodiment 15. The method of embodiment 14, wherein the dose of the compound for each administration is at least about 100 μg for a human or a comparable dose for an individual that is not a human.
Embodiment 16. The method of embodiment 15, wherein the dose of the compound for each administration is at least about 150 μg for a human or a comparable dose for an individual that is not a human.
Embodiment 17. The method of any one of embodiments 1-16, wherein the dose of the compound for each administration is no more than 2 mg for a human or a comparable dose for an individual that is not a human.
Embodiment 18. The method of any one of embodiments 1-17, wherein the dose of the compound for each administration is about 20 μg to about 2 mg for a human or a comparable dose for an individual that is not a human.
Embodiment 19. The method of embodiment 18, wherein the dose of the compound for each administration is about 20 μg to about 200 μg for a human or a comparable dose for an individual that is not a human.
Embodiment 20. The method of embodiment 19, wherein the dose of the compound for each administration is about 400 μg to about 2 mg for a human or a comparable dose for an individual that is not a human.
Embodiment 21. The method of any one of embodiments 1-20, wherein the composition is administered at a frequency of about once every two weeks to about three times a day.
Embodiment 22. The method of embodiment 21, wherein the composition is administered daily.
Embodiment 23. The method of embodiment 22, wherein the composition is administered daily at a dosage of about 20 μg to about 200 μg.
Embodiment 24. The method of embodiment 21, wherein the composition is administered weekly.
Embodiment 25. The method of embodiment 24, wherein the composition is administered weekly at a dosage of about 400 μg to about 2 mg.
Embodiment 26. The method of any one of embodiments 1-25, wherein the composition is administered intravenously, intraarterially, intraperitoneally, intravesicularly, subcutaneously, intrathecally, intrapulmonarily, intramuscularly, intratracheally, intraocularly, transdermally, orally, or by inhalation.
Embodiment 27. The method of embodiment 26, wherein the composition is administered orally.
Embodiment 28. The method of any one of embodiments 1-27, wherein the method further comprises administering a second agent.
Embodiment 29. The method of any one of embodiments 1-28, further comprising assessing estrogen level in the individual.
Embodiment 30. The method of any one of claims 1-29, wherein the insomnia is chronic insomnia.
Embodiment 31. A unit dosage form for treating insomnia comprising a) a composition comprising a compound of formula I, wherein the amount of the compound is in the range of about 20 μg to about 2 mg, and b) a pharmaceutical acceptable carrier.
Embodiment 32. Use of an effective amount of a composition comprising a compound of formula I (e.g., anordrin) for treating insomnia.
Embodiment 33. a composition comprising a compound of formula I (e.g., anordrin) for treating insomnia.
Embodiment 34. A kit comprising a) a composition comprising a compound of formula I, and b) instructions for using the kit for treating insomnia.

### EXAMPLES

The examples below are intended to be purely exemplary of the invention and should therefore not be considered to limit the invention in any way. The following examples and detailed description are offered by way of illustration and not by way of limitation.

### Example 1

Individuals who have taken anordrin (less than 1 mg) were self-assessed for the presence and the extent of insomnia before and after they took anordrin. Specifically, thirteen postmenopausal women took anordrin for about two weeks to a few months. Their sleep quality were evaluated with Athens Insomnia Scale (AIS). See FIG. 2.

TABLE 1

| | | before | after | | | Frequency of |
|---|---|---|---|---|---|---|
| # of AIS | Age | treatment | treatment | dosage | | the dosage |
| patient#1 | 81 | 20 | 5 | 0.25 mg | (per day) | Once per day or |
| | | | | Or 0.6 mg | (per week) | Once per week |
| patient#2 | 59 | 19 | 7 | 0.75 | mg | Once per week |
| patient#3 | 63 | 19 | 7 | 2 | mg | Once per week |
| patient#4 | 61 | 13 | 5 | 0.75 | mg | Once per week |
| patient#5 | 78 | 15 | 7 | 0.02-0.4 | mg | Once per day |
| patient#6 | 73 | 23 | 6 | 0.25 | mg | Once per day |
| patient#7 | 67 | 17 | 7 | 0.75 | mg | Once per day |
| patient#8 | 53 | 21 | 8 | 0.75 | mg | Once per week |
| patient#9 | 79 | 14 | 7 | 0.4 | mg | Once per day |
| patient#10 | 69 | 17 | 7 | 2 | mg | Once per week |
| patient#11 | 49 | 19 | 5 | 1 | mg | Once per week |
| patient#12 | 67 | 17 | 7 | 0.15 | mg | Once per day |
| patient#13 | 54 | 12 | 4 | 0.4 | mg | Once per day |
| Average | | 17.4 | 6.3 | | | |
| STDEV | | 3.2 | 1.2 | | | |
| p value | | | <0.0001 | | | |

As shown in FIG. 3 and Table 1, numbers of AIS for each patient significantly decreased after treatment of anordrin. The average number of AIS decreased by 63%. The results indicate that anordrin effectively improved sleep in these individuals.

Compound of formula A has also been tested and exhibited effectiveness (data not shown).

### Example 2

This Phase II clinical trial uses a randomized, double-blind, parallel-controlled design and is conducted in three clinical trial centers. A total of three drug groups are pre-set which are a) the 0.05 mg/day dosing group, b) the 0.1 mg/day dosing group, and c) the 0.15 mg/day dosing group as well as the placebo group. The drug intervention period is 8 weeks, and the drug intervention is followed up for 4 weeks. Primary efficacy endpoint includes vaginal detachment cell maturity index and dryness in postmenopausal women. Secondary efficacy endpoint includes quality in postmenopausal women, vaginal microecological index, vaginal pH in postmenopausal women, as well as the safety endpoint. The effectiveness and safety of different doses of α-anordrin tablets are preliminarily evaluated.

### Dosing Regimen

Group 1 (0.05 mg dose group): daily oral administration of one 0.05 mg α-anordrin enteric-coated tablet. Group 2 (0.1 mg dose group): daily oral administration of one 0.1 mg α-anordrin enteric-coated tablet. Group 3 (0.15 mg dose group): daily oral administration of one 0.15 mg α-anordrin enteric-coated tablet. Group 4: placebo group.

### Inclusion Criteria

Subjects must meet all of the following criteria in order to be eligible to enroll in the trial: 1) is of 45-80 years old, after surgery to remove the ovaries or it has been at least 12 months since the last menstrual period; 2) female patients have chronic insomnia, and the Athens Insomnia Scale (AIS) index is ≥7; 3) serum level of follicle stimulating hormone (FSH) is more than 40 U/L, estradiol (E2) is less than 35 pg/mL, and β-hCG is negative; 4) pelvic ultrasound examination shows endometrial thickness ≤0.4 cm, or the subject has been subject to hysterectomy; 5) meets the criteria for diagnosis and treatment of external vaginal atrophy: vaginal cell maturation index test results show ≤5% of vaginal upper cell percentage, vaginal pH is more than 5; 6) upon evaluation by the investigator, the trial can be completed in accordance with the program; 7) after fully understanding the purpose, content, process and possible benefits and risks of the trial, voluntarily participate in clinical research, sign informed consent, and comply with clinical research requirements.

### Endpoints

Efficacy endpoints include a) changes in vaginal exfoliation cell maturation index compared to baseline values; b) changes in insomnia severity index scores compared to baseline values; c) changes in vaginal microecological index score compared to baseline value; d) changes in vaginal pH compared to baseline values.

Safety endpoints include adverse events (including serious adverse events), laboratory tests and auxiliary examinations, vital signs, physical examination, etc.

### Preliminary Results

Table 2 shows the change of Athens Insomnia Scale (AIS) index of three patients after the treatment of anordrin. As shown, all three patients exhibit significantly improved sleeping quality after anordrin treatment with 0.05 mg/day. The results are consistent with what we saw in Example 1.

TABLE 2

| | | AIS number | AIS number | |
|---|---|---|---|---|
| # of AIS | Age | (before treatment) | (after treatment) | dosage |
| patient#1 | 56 | 12 | 4 | 0.05 mg/day |
| patient#2 | 69 | 17 | 5 | 0.05 mg/day |
| patient#3 | 65 | 19 | 5 | 0.05 mg/day |

### Example 3

It was reported that ATP levels in brain regions are stable during waking but exhibit a surge during the initial hours of sleep. This ATP surge displays a significant positive correlation with the intensity of EEG slow wave activity in non-rapid eye movement (NREM) sleep, a parameter of sleep depth and homeostatic need for sleep. See Chikahisa et al., Front Neurol. 2011; 2: 87. In this example, neuronal cells were treated with anordrin and ATP levels were measured to understand if anordrin treatment induced the ATP surge.

Materials and equipment include cell culture medium, FBS and Charcoal stripped FBS, ATP assay kit (Thermofisher) and rat primary neuronal cells (Wuhan servisebio).

Primary neuronal cells (2×10⁶) were grew in medium containing 10% FBS in 6 well plates for 24 hours. Then the medium was replaced with phenol red free medium contain 5% Charcoal stripped FBS for 24 hours. 10 nM anordrin or vechile were added in medium for 24 hours, and then cells were harvested. The fluorescence density of ATP was assayed using ATP assay kit following manufacture's instruction.

As shown in FIG. 5, anordrin significantly increased ATP level of neuronal cells, evidencing the positive impact of anordrin on sleep regulation.

1. A method of treating insomnia in an individual, comprising administering to the individual an effective amount of a composition comprising a compound of formula A: 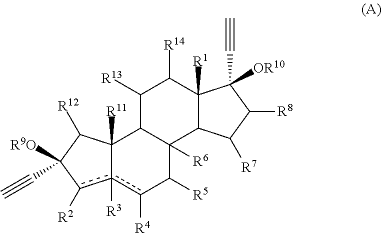
or a pharmaceutically acceptable salt or ester thereof, wherein:
a) [symbol] between the carbon bearing R² and the carbon bearing R³ represents a single bond, [symbol] between the carbon bearing R³ and the carbon bearing R⁴ represents a single bond, R² and R⁴ are independently selected from the group consisting of hydrogen, hydroxyl, mercaptan, keto, and C₁-C₃ alkyl, and R³ is selected from the group consisting of hydrogen, hydroxyl, mercaptan, and C₁-C₃ alkyl, or [symbol]
between the carbon bearing R² and the carbon bearing R³ represents a double bond, [symbol] between the carbon bearing R³ and the carbon bearing R⁴ represents a single bond, R² is selected from the group consisting of hydrogen, hydroxyl, mercaptan, and C₁-C₃ alkyl, R³ is absent, and R⁴ is selected from the group consisting of hydrogen, hydroxyl, mercaptan, keto, and C₁-C₃ alkyl, or [symbol]
between the carbon bearing R² and the carbon bearing R³ represents a single bond, [symbol] between the carbon bearing R³ and the carbon bearing R⁴ represents a double bond, R² is selected from the group consisting of hydrogen, hydroxyl, mercaptan, keto, and C₁-C₃ alkyl, R³ is absent, and R⁴ is selected from the group consisting of hydrogen, hydroxyl, mercaptan, and C₁-C₃ alkyl;
b) R¹ is C₁-C₃ alkyl;
c) R⁵, R⁷, R⁸, R¹², R¹³ and R¹⁴ are independently selected from the group consisting of hydrogen, hydroxyl, mercaptan, keto, and C₁-C₃ alkyl;
d) R⁶ is selected from the group consisting of hydrogen, hydroxyl, mercaptan, and C₁-C₃ alkyl;
e) R⁹ is selected from the group consisting of hydrogen and —C(═O)R^{9a};
f) R¹⁰ is selected from the group consisting of hydrogen and —C(═O)R^{10a};
g) R^{9a} and R^{10a} are independently selected from the group consisting of C₁-C₆ alkyl and C₂-C₆ alkenyl, wherein the C₁-C₆ alkyl and C₂-C₆ alkenyl are optionally substituted with —C(═O)OH; and
h) R¹¹ is selected from the group consisting of hydrogen and C₁-C₃ alkyl.

2. The method of claim 1, wherein R⁹ and R¹⁰ are independently hydrogen or —C(═O)CH₂CH₃.

3. The method of claim 1 or claim 2, wherein one or both of R¹ and R¹¹ are —CH₃.

4. The method of any one of claims 1-3, wherein any one or more of R², R³, R⁴, R⁵, R⁶, R⁷, R⁸, R¹², R¹³, and R¹⁴ are hydrogen.

5. The method of any one of claims 1-4, wherein the compound is anordrin.

6. The method of any one of claims 1-5, wherein the individual is a female.

7. The method of any one of claims 1-6, wherein the individual is a human.

8. The method of any one of claims 1-7, wherein the individual is at perimenopause stage, reaches menopause, is post menopause, or has been subject to a surgical removal of ovary.

9. The method of claim 8, wherein the individual is a postmenopausal woman.

10. The method of any one of claims 1-9, wherein the individual has a low level of estrogen.

11. The method of any one of claims 1-10, wherein the dose of the compound for each administration is at least about 20 μg for a human or a comparable dose for an individual that is not a human.

12. The method of claim 11, wherein the dose of the compound for each administration is at least about 50 μg for a human or a comparable dose for an individual that is not a human.

13. The method of claim 12, wherein the dose of the compound for each administration is at least about 100 μg for a human or a comparable dose for an individual that is not a human.

14. The method of claim 13, wherein the dose of the compound for each administration is at least about 150 μg for a human or a comparable dose for an individual that is not a human.

15. The method of any one of claims 1-14, wherein the dose of the compound for each administration is no more than 2 mg for a human or a comparable dose for an individual that is not a human.

16. The method of any one of claims 1-15, wherein the dose of the compound for each administration is about 20 μg to about 2 mg for a human or a comparable dose for an individual that is not a human.

17. The method of claim 16, wherein the dose of the compound for each administration is about 20 μg to about 200 μg for a human or a comparable dose for an individual that is not a human.

18. The method of claim 17, wherein the dose of the compound for each administration is about 400 μg to about 2 mg for a human or a comparable dose for an individual that is not a human.

19. The method of any one of claims 1-18, wherein the composition is administered at a frequency of about once every two weeks to about three times a day.

20. The method of claim 19, wherein the composition is administered daily.

21. The method of claim 20, wherein the composition is administered daily at a dosage of about 20 μg to about 200 μg.

22. The method of claim 19, wherein the composition is administered weekly.

23. The method of claim 22, wherein the composition is administered weekly at a dosage of about 400 μg to about 2 mg.

24. The method of any one of claims 1-23, wherein the composition is administered intravenously, intraarterially, intraperitoneally, intravesicularly, subcutaneously, intrathecally, intrapulmonarily, intramuscularly, intratracheally, intraocularly, transdermally, orally, or by inhalation.

25. The method of claim 24, wherein the composition is administered orally.

26. The method of any one of claims 1-25, wherein the method further comprises administering a second agent.

27. The method of any one of claims 1-26, further comprising assessing estrogen level in the individual.

28. The method of any one of claims 1-27, wherein the insomnia is chronic insomnia.

29. A unit dosage form for treating insomnia comprising a) a composition comprising a compound of formula I, wherein the amount of the compound is in the range of about 20 μg to about 2 mg, and b) a pharmaceutical acceptable carrier.

30. Use of an effective amount of a composition comprising a compound of formula I for treating insomnia.

31. A composition comprising a compound of formula I for treating insomnia.

32. A kit comprising a) a composition comprising a compound of formula I, and b) instructions for using the kit for treating insomnia.
